(19) United States
(12) Patent Application Publication
(10) Pub. No.: US 2024/0425889 A1
(43) Pub. Date: Dec. 26, 2024

(54) GENETIC ENGINEERING BACTERIUM FOR DE NOVO SYNTHESIS OF CIS,CIS-MUCONIC ACID BY TAKING GLUCOSE AS SUBSTRATE AND APPLICATIONS THEREOF

(71) Applicant: Beijing University of Chemical Technology, Beijing (CN)

(72) Inventors: Tianwei Tan, Beijing (CN); Menglei Li, Beijing (CN)

(21) Appl. No.: 18/825,195

(22) Filed: Sep. 5, 2024

Related U.S. Application Data
(63) Continuation of application No. PCT/CN2024/095637, filed on May 28, 2024.

(30) Foreign Application Priority Data
Sep. 27, 2023 (CN) 202311253745.4

(51) Int. Cl. C12P 7/44; C12N 9/02; C12N 9/10; C12N 9/88; C12N 9/90; C12N 15/52; C12N 15/77

(52) U.S. Cl. CPC C12P 7/44; C12N 9/0069; C12N 9/1022; C12N 9/1085; C12N 9/88; C12N 9/90; C12N 15/52; C12N 15/77; C12Y 113/11001; C12Y 202/01001; C12Y 401/01061; C12Y 401/01063; C12Y 402/0101; C12Y 402/03004

(57) ABSTRACT

The present invention discloses a genetic engineering bacterium for de novo synthesis of cis,cis-muconic acid by taking glucose as a substrate and applications thereof, and belongs to the technical field of genetic recombination and metabolic engineering. The genetic engineering bacterium for de novo synthesis of cis,cis-muconic acid (MA) by taking glucose as the substrate disclosed in the present invention is modified with chassis microbes, and includes recombinant Corynebacterium glutamicum for a cis,cis-muconic acid pathway construction module and an intermediate high-yield module. Production capacity of strains is greatly improved; MA of 90.2 g/L is finally obtained in fermentation liquor; and possibilities are provided for green and low-cost production of numerous chemicals such as adipic acid and nylon-66.

### SEQUENCE LISTING

The substitute sequence listing is submitted as a XML file filed via EFS-Web, with a file name of “Sequence_Listing.XML”, a creation date of Sep. 3, 2024, and a size of 96,896 bytes. The substitute sequence Listing filed via EFS-Web is a part of the specification and is incorporated in its entirety by reference herein.

### TECHNICAL FIELD

The present invention relates to the technical field of genetic recombination and metabolic engineering, and more particularly relates to a genetic engineering bacterium for de novo synthesis of cis,cis-muconic acid by taking glucose as a substrate and applications thereof.

### BACKGROUND

cis,cis-muconic acid (MA) is a C6 compound of which two ends have two carboxy groups and the middle has two double bonds. It has a molecular formula of C₆H₆O₄ and a molecular weight of 142.11, is an industrial platform chemical compound for producing essential chemicals such as nylon-66, adipic acid and polyurethane, and is an important potential raw material for preparing functional resins, medicines and agricultural chemicals. Particularly the MA plays an important role in producing the adipic acid while serving as a raw material. Consumption of adipic acid has already exceeded 4 million tonnages all over the world; the consumption of the adipic acid also exceeds 1 million tonnages in China; and the quantity demanded is increasing with the development of economy. Traditional MA production is chemical synthesis, and mainly refers to production performed by utilizing nonrenewable petrochemical resource derivatives as raw materials through highly corrosive or irritating solvents such as peracetic acid and acetic acid. Such a method has high time consumption and great pollution, and mismatches with the goal of sustainable development in today's world. Therefore, developing an environment-friendly MA production method attracts more and more attentions. With the gradual maturity of genetic manipulation means and metabolic engineering technologies, the research focus of MA production has gradually approached to microbial conversion. A biological conversion method does not need any catalyst, solvent or oxidant, and is a clean and high-harmlessness production process. The biological conversion may effectively alleviate numerous adverse effects brought by chemical synthesis of the MA, but a concentration of the MA produced by a microbial method and production efficiency need to be further improved.

Different microbes have different endogenous metabolic properties, and mainly include two manners such as biological conversion of aromatic compounds and microbial fermentation of saccharides in the microbial conversion. For example, the microbes such as Pseudomonas putida and Rhodococcus opacus may metabolize some aromatic compounds through optimized expression of p-hydroxybenzoic acid-3-hydroxylase in strains; and MA of 40 g/L is produced in fed-batch fermentation by taking p-coumaric acid as a substrate (Kuatsjah E, et al. Debottlenecking 4-hydroxybenzoate hydroxylation in Pseudomonas putida KT2440 improves muconate productivity from p-coumarate). Vanillin dehydrogenase is introduced by knocking out vanillin dehydrogenase genes in Rhodococcus opacus by Jin Mingjie, et al., and MA of 4.3 g/L is obtained, thereby achieving conversion from the vanillin to the MA. (Jin, et al. Deciphering the metabolic distribution of vanillin in Rhodococcus opacus during lignin valorization). However, most of the benzene raw materials used in current production are derived from petroleum resources and belong to nonrenewable raw materials, while direct production of the MA by utilizing a simple carbon source such as glucose has more important significance to some extent. Most of the conventional model organisms such as Saccharomyces cerevisiae and Escherichia coli are modified to directly produce the MA by utilizing the glucose. In researches of Wang, et al., by changing shikimate pathway flux in Saccharomycetes and increasing phosphoenolpyruvate supply, production of MA of 20.8 g/L is achieved in a 10 L fermentation tank (Wang G, et al. An integrated yeast-based process for cis, cis-muconic acid production). However, Corynebacterium glutamicum is a microbe having high fermentation capacity, and has been applied to industrial production of amino acids such as L-glutamic acid and L-lysine. Moreover, metabolically, the MA is just an intermediate of the own β-ketoadipic acid pathway. The Corynebacterium glutamicum has the congenital advantage of producing the MA, and is an ideal host for MA production in a biosynthetic pathway. Since bulk chemicals have relatively low values, increasing the product concentration and synthetic efficiency is the key to reduce the cost. To facilitate more efficient production of a target product in a cell factory, production strains must be continuously optimized and modified to increase the target product conversion efficiency by continuously utilizing many means such as molecular biological techniques and fermentation control, thereby creating a possibility for efficient and low-cost production.

Therefore, providing a genetic engineering bacterium for de novo synthesis of cis,cis-muconic acid by taking glucose as a substrate and applications thereof is a problem that urgently needs to be solved by those skilled in the art.

### SUMMARY

In view of this, the present invention provides a genetic engineering bacterium for de novo synthesis of cis,cis-muconic acid (MA) by taking glucose as a substrate and applications thereof. With respect to problems existing in de novo synthesis of MA in a current biological method, a biological pathway for efficient de novo synthesis of MA is developed, and a recombinant Corynebacterium glutamicum that can be used for producing the MA by more efficiently utilizing glucose is constructed.

To achieve the above purpose, the present invention adopts the following technical solution:

A genetic engineering bacterium for de novo synthesis of cis,cis-muconic acid by taking glucose as a substrate is provided. The genetic engineering bacterium is modified with chassis microbes, and includes recombinant Corynebacterium glutamicum for a cis,cis-muconic acid pathway construction module and an intermediate high-yield module.

Modification of the chassis microbes is as follows: the Corynebacterium glutamicum serves as an original strain; and pyk(cgl2089)(pyruvate kinase), aroE(cgl1835) (shikimate5-dehydrogenase), pcaG/H(cgl2630, cgl2631) (protocatechuate 3,4-dioxygenase subunit beta (pcaH), protocatechuate 3,4-dioxygenase subunit alpha (pcaG)), and catB(cgl2635) (chloromuconate cycloisomerase) genes are knocked out.

The cis,cis-muconic acid pathway construction module expresses protocatechuate decarboxylase genes, UbiX-like flavin prenyltransferase genes, 4-hydroxybenzoate decarboxylase, subunit D genes and catechol 1,2-dioxygenase genes.

The intermediate high-yield module expresses 3-dehydroquinate synthase genes, 3-dehydroquinate dehydratase genes, phosphate isomerase genes and transketolase genes.

Further, the protocatechuate decarboxylase genes are Klebsiella Pneumoniae-derived protocatechuate decarboxylase genes aroY.

The UbiX-like flavin prenyltransferase genes include Escherichia coli-derived UbiX-like flavin prenyltransferase genes kpdB, and Klebsiella pneumoniae-derived UbiX-like flavin prenyltransferase genes kpdB.

The 4-hydroxybenzoate decarboxylase, subunit D genes include Escherichia coli-derived 4-hydroxybenzoate decarboxylase, subunit D genes kpdD, and Klebsiella pneumoniae-derived 4-hydroxybenzoate decarboxylase, subunit D genes kpdD.

The catechol 1,2-dioxygenase genes include Pseudomonas putida-derived catechol 1,2-dioxygenase genes catA and Corynebacterium glutamicum endogenous catechol 1,2-dioxygenase genes catA.

The 3-dehydroquinate synthase is a Corynebacterium glutamicum endogenous 3-dehydroquinate synthase gene aroB.

The 3-dehydroquinate dehydratase genes are Corynebacterium glutamicum endogenous 3-dehydroquinate dehydratase genes aroD.

The phosphate isomerase genes are Corynebacterium glutamicum endogenous phosphate isomerase genes qsuB.

A nucleotide sequence of the Klebsiella pneumoniae-derived protocatechuate decarboxylase genes is shown as SEQ ID NO. 37.

A nucleotide sequence of the Escherichia coli-derived UbiX-like flavin prenyltransferase genes kpdB is shown as SEQ ID NO. 68.

A nucleotide sequence of the Klebsiella pneumoniae-derived UbiX-like flavin prenyltransferase genes kpdB is shown as SEQ ID NO. 38.

A nucleotide sequence of the Escherichia coli-derived 4-hydroxybenzoate decarboxylase, subunit D genes kpdD is shown as SEQ ID NO. 69.

A nucleotide sequence of the Klebsiella pneumoniae-derived 4-hydroxybenzoate decarboxylase, subunit D genes kpdD is shown as SEQ ID NO. 39.

A nucleotide sequence of the Pseudomonas putida-derived catechol 1,2-dioxygenase genes catA is shown as SEQ ID NO. 76.

A nucleotide sequence of the Corynebacterium glutamicum endogenous catechol 1,2-dioxygenase genes catA is shown as SEQ ID NO. 35.

A nucleotide sequence of the Corynebacterium glutamicum endogenous 3-dehydroquinate synthase gene aroB is shown as SEQ ID NO. 51.

A nucleotide sequence of the Corynebacterium glutamicum endogenous 3-dehydroquinate dehydratase genes aroD is shown as SEQ ID NO. 52.

A nucleotide sequence of the Corynebacterium glutamicum endogenous phosphate isomerase genes qsuB is shown as SEQ ID NO. 50.

A nucleotide sequence of the transketolase genes tkt is shown as SEQ ID NO. 53.

Preferably, the protocatechuate decarboxylase genes are the Klebsiella pneumoniae-derived protocatechuate decarboxylase genes aroY.

The UbiX-like flavin prenyltransferase genes are the Klebsiella pneumoniae-derived UbiX-like flavin prenyltransferase genes kpdB.

The 4-hydroxybenzoate decarboxylase, subunit D genes are the Klebsiella pneumoniae-derived 4-hydroxybenzoate decarboxylase, subunit D genes kpdD.

The catechol 1,2-dioxygenase genes are the Corynebacterium glutamicum endogenous catechol 1,2-dioxygenase genes catA.

The 3-dehydroquinate synthase is the Corynebacterium glutamicum endogenous 3-dehydroquinate synthase gene aroB.

The 3-dehydroquinate dehydratase genes are the Corynebacterium glutamicum endogenous 3-dehydroquinate dehydratase genes aroD.

The phosphate isomerase genes are the Corynebacterium glutamicum endogenous phosphate isomerase genes qsuB.

Further, the intermediate high-yield module further expresses phospho-2-dehydro-3-deoxyheptonate aldolase genes.

A nucleotide sequence of the phospho-2-dehydro-3-deoxyheptonate aldolase genes is shown as SEQ ID NO. 81.

Further, a construction method of a genetic engineering bacterium for de novo synthesis of cis,cis-muconic acid by taking glucose as a substrate includes the following steps:
(1) taking Corynebacterium glutamicum ATCC13032 as an original strain, and knocking out pyk(cgl2089), aroE(cgl1835), pcaG/H(cgl2630, cgl2631) and catB(cgl2635) genes to obtain modified Corynebacterium glutamicum; and
(2) expressing the cis,cis-muconic acid pathway construction module and the intermediate high-yield module in the modified Corynebacterium glutamicum.

Further, the present invention provides an application of the genetic engineering bacterium or the method in production of cis,cis-muconic acid.

Further, the present invention provides an application of the genetic engineering bacterium or the method in yield increase of the cis,cis-muconic acid.

Further, the present invention provides a method for producing cis,cis-muconic acid, wherein fermentation is performed by utilizing the genetic engineering bacterium or a genetic engineering bacterium constructed by the method.

Thus, the present invention provides a biological pathway for synthesizing cis,cis-muconic acid (FIG. 1):

In the Corynebacterium glutamicum, 3 major decomposition pathways in a glucose production pathway are knocked out by virtue of chassis modification; and deletion in MA production with the glucose and low-activity enzyme genes and related proteins thereof are expressed, thereby enhancing key enzyme genes in a core pathway.

The major decomposition pathways in MA production with the glucose are as follows: aroE(cgl1835) (shikimate 5-dehydrogenase) is knocked out to decrease shikimate flux; pcaG/H(cgl2630, cgl2631) (protocatechuate 3,4-dioxygenase subunit beta (pcaH), protocatechuate 3,4-dioxygenase subunit alpha (pcaG)) are knocked out to block transformation to 3-carboxymuconic acid; catB(cgl2635) (chloromuconate cycloisomerase) is knocked out to block muconic acid from being decomposed into muconolactone; and the major decomposition pathways in MA production are knocked out, thereby obtaining higher flux in MA production and achieving higher enrichment of the MA.

The expression of the deletion in MA production with the glucose and low-activity enzyme genes and related proteins thereof is as follows: by expressing aroY (protocatechuate decarboxylase), kpdB (UbiX-like flavin prenyltransferase), kpdD (4-hydroxybenzoate decarboxylase, subunit D) and catA (catechol 1,2-dioxygenase) of different sources, the MA production pathway in the strain is constructed.

The enhancement of the key enzyme genes in the core pathway is as follows: by knocking out pyk (cgl2089) (pyruvate kinase) and expressing tkt (transketolase), accumulation of precursor substances phosphoenolpyruvate (PEP) and erythrose 4-phophate (E4P) synthesized by 3-deoxy-D-arabinoheptulosonate-7-phosphate (DAHP) in the shikimate pathway is respectively increased; and the flux in the production pathway is increased by increasing flux of the PEP and the E4P, thereby facilitating production of the MA.

The enhancement of the key enzyme genes in the core pathway is further as follows: endogenous aroG (phospho-2-dehydro-3-deoxyheptonate aldolase), aroB (3-dehydroquinate synthase), aroD (shikimate/quinate dehydratase) and qsuB (phosphate isomerase) are over-expressed by utilizing a combinational metabolic engineering strategy; and by virtue of over-expression of key intermediate production genes, an intermediate conversion degree is improved, thereby obtaining more MA accumulation.

Through the above technical solutions, compared with the prior art, the present invention provides the genetic engineering bacterium for de novo synthesis of cis,cis-muconic acid by taking glucose as the substrate and applications of the genetic engineering bacterium, and specifically provides a more efficient recombinant Corynebacterium glutamicum for production of MA by utilizing the glucose. By knocking out the major decomposition pathways in the production pathway and expressing the endogenous deletion of the strain and the low-activity enzyme genes and proteins thereof, the production pathway in the strain is constructed. Then, by increasing metabolic flux of the production pathway and enhancing the key enzyme genes in the core pathway, the production capacity of the strain is greatly improved; MA of 90.2 g/L is finally obtained in fermentation liquor and is the highest record of the MA produced by taking glucose or (and) glycerin as the substrate at present; and the product has wide application prospects. Moreover, possibilities are provided for green and low-cost production of numerous chemicals such as adipic acid and nylon-66.

### DESCRIPTION OF DRAWINGS

To more clearly describe the technical solutions in the embodiments of the present invention or in the prior art, the drawings required to be used in the description of the embodiments or the prior art will be simply presented below. Obviously, the drawings in the following description are merely embodiments of the present invention, and for those ordinary skilled in the art, other drawings can also be obtained according to the provided drawings without contributing creative labor.

FIG. 1 is a biological synthesis pathway of cis,cis-muconic acid in the present invention;

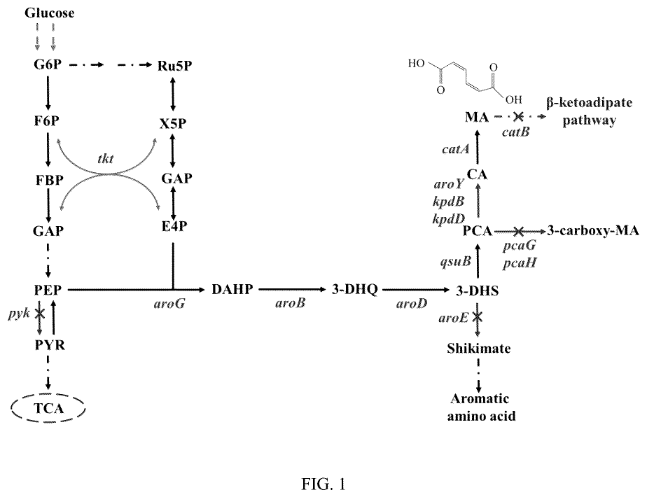

FIG. 2 shows growth and metabolism of a strain MA1 in fermentation of different systems and MA production situations in the present invention, wherein A: a fermentation system in a 50 mL shaking flask; B: a fermentation system in a 5 L fermentation tank;

FIG. 3 shows growth and metabolism of a strain MA2 in fermentation of a 50 mL shaking flask and MA production situations in the present invention;

FIG. 4 shows growth and metabolism of a strain MA3 in fermentation of a 50 mL shaking flask and MA production situations in the present invention;

FIG. 5 shows growth and metabolism of a strain MA4 in fermentation of a 50 mL shaking flask and MA production situations in the present invention.

### DETAILED DESCRIPTION

Technical solutions in the embodiments of the present invention are described clearly and fully below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

LBHIS medium: 5.0 g/L of peptone, 2.5 g/L of yeast powder, 5.0 g/L of NaCl, 18.5 g/L of brain heart infusion (BHI) and 91.0 g/L of sorbitol. 1.8-2% of agar is added into a corresponding LBHIS solid medium.

LBG medium: 10.0 g/L of peptone, 5.0 g/L of yeast powder, 10.0 g/L of NaCl and 20.0 g/L of glucose.

EPO medium: 10.0 g/L of peptone, 5.0 g/L of yeast powder, 10.0 g/L of NaCl, 30.0 g/L of glycine and 10.0 g/L of Tween80.

30% sucrose medium: 10.0 g/L of peptone, 5.0 g/L of yeast powder, 10.0 g/L of NaCl and 300.0 g/L of sucrose.

20% sucrose solid medium: 10.0 g/L of peptone, 5.0 g/L of yeast powder, 10.0 g/L of NaCl, 200.0 g/L of sucrose and 15.0 g/L of agar.

Fermentation medium: 65.0 g/L of glucose, 5.0 g/L of urea, 8.0 g/L of corn extract powder, 4×10⁻⁴ g/L of biotin, 4×10⁻⁴ g/L of VB₁ biotin, 1.0 g/L of K₂HPO₄, 1.0 g/L of KH₂PO₄, 29.4 mg/L of CaCl₂·2H₂O, 1.2325 g/L of MgSO₄ 7H₂O and 0.2% of a trace element solution. (a preparation method of the trace element solution includes steps: weighing 1 g of FeSO₄·7H₂O, 1 g of MnSO₄·H₂O, 0.1 g of ZnSO₄·7H₂O, 0.2 g of CuSO₄ and 0.002 g of NiCl₂·6H₂O, adding water to obtain a fixed volume of 100 mL; then adding 100 μl of concentrated hydrochloric acid to regulate the pH value; filtering the bacterium with a filtration membrane; and adding 0.2% into a fermentation medium system.)

### Embodiment 1

A construction method of a genetic engineering bacterium for de novo synthesis of cis,cis-muconic acid by taking glucose as a substrate includes the following steps:

(1) Genome DNA of Corynebacterium glutamicum ATCC13032 was taken as a template; catB-up was amplified by using a primer catB-up-F/catB-up-R (as shown in SEQ ID NO. 1); and catB-down was amplified by using a primer catB-down-F/catB-down-R (as shown in SEQ ID NO. 2), so as to obtain upstream and downstream homologous arms of which the catB was knocked out. Primer sequences were as follows:

catB-up-F:
SEQ ID NO. 3
5′-CTAGACTTTTTCCAGAGCTTTTTGGAACAGCTTG-3′;;
catB-up-R:
SEQ ID NO. 4
5′-CCAGAAAGCCATAGAAAAAGGAGAATTATCGATGCTGTTTC
TAGCACG-3′;;
catB-down-F:
SEQ ID NO. 5
5′-TTTTTCTATGGCTTTCTGGTTAAGTGGGAAA-3′;;
catB-down-R:
SEQ ID NO. 6
5′-TTATCGAGTTCAGCCGATCACAAAGATTTTTC-3′;.

Gene amplification system: 25 μL of Primestar (Takara), 2 μL of a forward primer, 2 μL of a reverse primer, 1 μL of a template and 20 μL of ddH₂O.

Gene amplification procedures: pre-denaturation at 98° C. for 3 minutes; denaturation at 98° C. for 10 seconds; annealing at 57° C. for 30 seconds; extension at 72° C. by 10 s/kb, totaling 35 cycles; and extension at 72° C. for 10 minutes.

The PCR product was subjected to agarose gel electrophoresis and product recovery to obtain gene segments catB-up and catB-down.

A plasmid vector pk18mobsacb was linearized with a primer pK18-catBF/pK18-catBR to obtain a vector pk-catB, wherein primer sequences were as follows:

pK18-catBF:
SEQ ID NO. 7
5′-ATCGGCTGAACTCGATAAAAGCTTGGCACTGGCCG-3′;;
pK18-catBR:
SEQ ID NO. 8
5′-CAAAAAGCTCTGGAAAAAGTCTAGAGGATCCCCGGGTACCG-3′;.

3 μL of catB-up, 3 μL of catB-down, 4 μL of a vector pK-catB and 10 μL of Gibson ligase were added into a PCR tube, wherein a ligation temperature was 50° C.; ligation time was 15 min; and a total volume of the system was 20 μL.

The total 20 μL Gibson-ligated ligation system was transformed into E. coli Trans10 commercial competent cells (TransGen Biotech) for culture and retention. The transformation process was strictly operated according to the instructions as follows: after cultured at 37° C. for 1 h, the ligation system was coated onto an LB plate (containing 50 μg/mL of kanamycin); then the system was cultured at 37° C. for 12 h; and 10-20 single colonies were selected to perform PCR amplification and DNA sequencing verification. A primer used in PCR amplification and DNA sequencing verification was the above catB-up-F/catB-down-R.

An accurate single colony was selected and then named as Escherichia coli EC001; a plasmid was named as pK18-catB and subjected to expanding propagation to perform plasmid extraction so as to obtain a plasmid pK18-catB; and then the plasmid was electro-transformed into Corynebacterium glutamicum ATCC13032 competent cells.

Preparation of the Corynebacterium glutamicum ATCC13032 competent cells was as follows: a glycerin-preserved strain of Corynebacterium glutamicum ATCC13032 was subjected to streak inoculation on an LBHIS plate; the strain was cultured in an incubator at 30° C. until the colony was clear; the colony was picked from the plate and inoculated into an LBHIS test-tube fluid medium for culture for 12 h; then the culture solution was inoculated into an EPO medium at an initial OD₆₀₀ of about 0.3; the solution was continuously cultured until the OD₆₀₀ was 0.9; the bacterium solution was added into a centrifuge tube; the centrifuge tube was placed in an ice bath for 15 min; the bacterium solution was frozen and centrifuged at 4500 rpm for 10 min to collect the thallus (separately filled in 1.5 mL centrifuge tubes); the thallus was resuspended with 100 μL of pre-cooled 10% glycerin; the above centrifugation was continuously repeated for three times; and the thallus was resuspended with 100 μL of 10% sterile glycerin after washing completion, thereby obtaining the Corynebacterium glutamicum ATCC13032 competent cells.

The electrotransformation method was as follows: 2-4 μL of the plasmid was added into competent cells of each tube and placed in an ice bath for 10 min; the mixed solution was transferred into a pre-cooled electric shock cup, and electric shock was conducted under conditions of 1.8 kv, an electric shock rate of 5 ms, 50 μF and 100Ω; a 800 μL LBHIS medium was immediately added after electric shock completion; then the solution was placed in a water bath at 46° C. for 6 min and then cultured at 30° C. for 2-3 h; the solution was coated on a solid LBHIS plate containing 50 μg/mL kanamycin; and the solution was cultured at 30° C. for 24-36 h for verification.

For a gene knockout strain, several single colonies growing on the above plate needed to be subjected to sacB gene verification, wherein verification primers were as follows:

sacB-F:
SEQ ID NO. 9
5′-CTCAAGCGTTTGCGAAAGAAACG-3′;;
sacB-R:
SEQ ID NO. 10
5′-GAGTCAGTGAACAGGTACCATTTGCC-3′;.

The accurately verified strain was inoculated into a 30% sucrose medium to be cultured for 24 h; after the bacterium solution was turbid, the strain was subjected to streak culture on a 20% sucrose medium; colony PCR of the sacB gene was continuously performed; then a single colony in which the sacB gene was not amplified was the accurate colony; the thallus was inoculated into an LBHIS fluid medium for culture; the thallus was purified on an LBHIS solid plate after the solution was turbid; and verification was performed by using forward and reverse primers catB-up-F/catB-up-R and catB-down-F/catB-down-R of the target gene. After confirmed, the strain was the target gene knockout strain and may be preserved for later use. The strain was named as Corynebacterium glutamicum GM1.

(2) Genome DNA of Corynebacterium glutamicum ATCC13032 was taken as a template; aroE-up was amplified by using a primer aroE-up-F/aroE-up-R (as shown in SEQ ID NO. 11); and aroE-down was amplified by using a primer aroE-down-F/aroE-down-R (as shown in SEQ ID NO. 12), so as to obtain upstream and downstream homologous arms of which the aroE was knocked out. Primer sequences were as follows:

aroE-up-F:
SEQ ID NO. 13
5′-GTCGCTCACCGCGATTTTAGAGTGGC-3′;;
aroE-up-R:
SEQ ID NO. 14
5′-GATGTGACTAAATCTTAGTGACTATTTACATGGGTGG-3′;;
aroE-down-F:
SEQ ID NO. 15
5′-GTCACTAAGATTTAGTCACATCCGAGAGCCGAGTACCG-3′;;
aroE-down-R:
SEQ ID NO. 16
5′-TCTCTGCTTGAGACTTAAGCGTTATCCG-3′;.

A plasmid vector pk18mobsacb was linearized with a primer pK18-aroEF/pK18-aroER to obtain a vector pk-aroE, wherein primer sequences were as follows:

pK18-aroEF:
SEQ ID NO. 17
5′-CTTAAGTCTCAAGCAGAGAGACCTGCAGGCATGCAAGC-3′;;
pK18-aroER:
SEQ ID NO. 18
5′-TAAAATCGCGGTGAGCGACTCTAGAGGATCCCCGGGTAC-3′;.

The gene amplification system and gene amplification procedures were the same as above; vectors aroE-up, aroE-down and pK-aroE were obtained; ligation and transformation verification manners were the same as above; Escherichia coli EC002 was obtained; the plasmid was named as pK18-aroE; and through expanding propagation, the plasmid was extracted to obtain a plasmid pK18-aroE.

The plasmid pK18-aroE was electro-transformed into Corynebacterium glutamicum GM1 competent cells. Preparation of the competent cells, electro-transformation, sacB verification and culture of the sucrose medium were the same as above. The aroE knockout verification primer was the above aroE-up-F/aroE-down-R.

Through verification of the verification primer, a strain of which the aroE was successfully knocked out was inoculated into an LBHIS fluid medium for culture; and the strain was subjected to streak culture on an LBHIS solid medium plate for purification and re-verification. After confirmed, the strain may be preserved for later use, and was named as Corynebacterium glutamicum GM2.

(3) Genome DNA of Corynebacterium glutamicum ATCC13032 was taken as a template; pcaG/H-up was amplified by using a primer pcaG/H-up-F/pcaG/H-up-R (as shown in SEQ ID NO. 19); and pcaG/H-down was amplified by using a primer pcaG/H-down-F/pcaG/H-down-R (as shown in SEQ ID NO. 20), so as to obtain upstream and downstream homologous arms of which the pcaG/H was knocked out. Primer sequences were as follows:

pcaG/H-up-F:
SEQ ID NO. 21
5′-AACGTTGACGGTGATGCCATC-3′;;
pcaG/H-up-R:
SEQ ID NO. 22
5′-ATTGACCCGATCTTTATACTCCGACCTTG-3′;;
pcaG/H-down-F:
SEQ ID NO. 23
5′-GAGTATAAAGATCGGGTCAATGCGAGACCTTTCT
GCGTCTAGTG-3′;;
pcaG/H-down-R:
SEQ ID NO. 24
5′-CCATCGCATTGCCGAAAAGCTG-3′;.

A plasmid vector pk18mobsacb was linearized with a primer pK18-pcaF/pK18-pcaR to obtain a vector pk-pca, wherein primer sequences were as follows:

pK18-pcaF:
SEQ ID NO. 25
5′-TTCGGCAATGCGATGGGACCTGCAGGCATGCAAGC-3′;;
pK18-pcaR:
SEQ ID NO. 26
5′-ATCACCGTCAACGTTGACTCTAGAGGATC-3′;.

The gene amplification system and gene amplification procedures were the same as above; vectors pcaG/H-up, pcaG/H-down and pk-pca were obtained; ligation and transformation verification manners were the same as above; Escherichia coli EC003 was obtained; the plasmid was named as pK18-pca; and through expanding propagation, the plasmid was extracted to obtain a plasmid pK18-pca.

The plasmid pK18-pca was electro-transformed into Corynebacterium glutamicum GM2 competent cells. Preparation of the competent cells, electro-transformation, sacB verification and culture of the sucrose medium were the same as above. The pcaG/H knockout verification primer was the above pcaG/H-up-F/pcaG/H-down-R.

Through verification of the verification primer, a strain of which the pcaG/H was successfully knocked out was inoculated into an LBHIS fluid medium for culture; and the strain was subjected to streak culture on an LBHIS solid medium plate for purification and re-verification. After confirmed, the strain may be preserved for later use, and was named as Corynebacterium glutamicum GM3.

(4) Genome DNA of Corynebacterium glutamicum ATCC13032 was taken as a template; pyk-up was amplified by using a primer pyk-up-F/pyk-up-R (as shown in SEQ ID NO. 27); and pyk-down was amplified by using a primer pyk-down-F/pyk-down-R (as shown in SEQ ID NO. 28), so as to obtain upstream and downstream homologous arms of which the pyk was knocked out. Primer sequences were as follows:

pyk-up-F:
SEQ ID NO. 29
5′-CTCTAGAGTCCAACAGAGGTGCCGTTGTCAAAG-3′;;
pyk-up-R:
SEQ ID NO. 30
5′-GGCTCGCTTAAATCTTTCAAAAAATGCGTTGACAC-3′;;
pyk-down-F:
SEQ ID NO. 31
5′-GAAAGATTTAAGCGAGCCCATAAGCCTAGTACGTCATTCC-3′;;
pyk-down-R:
SEQ ID NO. 32
5′-CAGGTCGTCCTTGATGCAGCGATCGTG-3′;.

A plasmid vector pk18mobsacb was linearized with a primer pK18-pykF/pK18-pykR to obtain a vector pk-pyk, wherein primer sequences were as follows:

pK18-pykF:
SEQ ID NO. 33
5′-CATCAAGGACGACCTGCAGGCATGCAAGC-3′;;
pK18-pykR:
SEQ ID NO. 34
5′-CTCTGTTGGACTCTAGAGGATCCCCGGGTAC-3′;.

The gene amplification system and gene amplification procedures were the same as above; vectors pyk-up, pyk-down and pk-pyk were obtained; ligation and transformation verification manners were the same as above; Escherichia coli EC004 was obtained; the plasmid was named as pK18-pyk; and through expanding propagation, the plasmid was extracted to obtain a plasmid pK18-pyk.

The plasmid pK18-pyk was electro-transformed into Corynebacterium glutamicum GM3 competent cells. Preparation of the competent cells, electro-transformation, sacB verification and culture of the sucrose medium were the same as above. The pyk knockout verification primer was the above pyk-up-F/pyk-down-R.

Through verification of the verification primer, a strain of which the pyk was successfully knocked out was inoculated into an LBHIS fluid medium for culture; and the strain was subjected to streak culture on an LBHIS solid medium plate for purification and re-verification. After confirmed, the strain may be preserved for later use, and was named as Corynebacterium glutamicum GM4.

(5) Genome DNA of Corynebacterium glutamicum ATCC13032 was taken as a template; the segment catA was amplified by using a primer CG-catA-F/CG-catA-R (as shown in SEQ ID NO. 35); and Corynebacterium glutamicum derived catA with seamless cloning homologous arms (ribosome bind sites, as shown in SEQ ID NO. 36) ligated with the plasmid was obtained.

Genome DNA of Kleber pneumonia subspecies was taken as a template; the segment aroY was amplified by using a primer aroY-F/aroY-R (as shown in SEQ ID NO. 37); and a Kleber pneumonia-derived aroY segment with seamless cloning homologous arms in ligation with the catA and with RBS was obtained.

Genome DNA of Kleber pneumonia subspecies was taken as a template; the segment kpdB was amplified by using a primer KP-kpdB-F/KP-kpdB-R (as shown in SEQ ID NO. 38); and a Kleber pneumonia-derived kpdB segment with seamless cloning homologous arms in ligation with the aroY was obtained.

Genome DNA of Kleber pneumonia subspecies was taken as a template; the segment kpdD was amplified by using a primer KP-kpdD-F/KP-kpdD-R (as shown in SEQ ID NO. 39); and a Kleber pneumonia-derived kpdD segment with seamless cloning homologous arms in ligation with the kpdB and the plasmid and with RBS was obtained.

Primer sequences are as follows:

CG-catA-F:
SEQ ID NO. 40
5′-CATATGACTTCAGCTGAACAGATCGTTGATC-3′;;
CG-catA-R:
SEQ ID NO. 41
5′-CTAGTCTTCCTTATCCAGGACGAATGGG-3′;;
aroY-F:
SEQ ID NO. 42
5′-CCTGGATAAGGAAGACTAGAAGGAGGATATACATATGACCGCA
CCGATTCAGGATC-3′;;
aroY-R:
SEQ ID NO. 43
5′-ATCCCAATAATCAGTTTCATATGTATATCCTCCTTTTATTTTGCG
CTACCCTGGTTTTTTTCC-3′;;
KP-kpdB-F:
SEQ ID NO. 44
5′-CATATGAAACTGATTATTGGGATGACGGGG-3′;;
KP-kpdB-R:
SEQ ID NO. 45
5′-TTATTCGATCTCCTGTGCAAATTGTTCT-3′;;
KP-kpdD-F:
SEQ ID NO. 46
5′-GCACAGGAGATCGAATAAAAGGAGGATATACATATGATTT
GTCCACGTTGCGC-3′;;
KP-kpdD-R:
SEQ ID NO. 47
5′-GATTAACGCTTATCTTCCGGCAATAGCG-3′;.

A plasmid vector pEC-XK99E was linearized with a primer PEC-1-1F/PEC-1-1R to obtain a vector PEC, wherein primer sequences were as follows:

PEC-1-1F:
SEQ ID NO. 48
5′-CGGAAGATAAGCGTTAATCTAGAGTCGACCTGCAGGCATG-3′;;
PEC-1-1R:
SEQ ID NO. 49
5′-GTTCAGCTGAAGTCATATGTATATCCTCCTTGAGCTCGAAT
TCTTCTGTTTCCTGTG-3′;.

The gene amplification system and gene amplification procedures were the same as above; vectors catA, aroY, kpdB, kpdD and pEC-1 were obtained; and 1.5 μl of catA, 1.5 μl of aroY, 1.5 μl of kpdB, 1.5 μl of kpdD and 4 μl of pEC of different sources and 10 μl of Gibson ligase were added into PCR tubes. Ligation and transformation manners were the same as above; several selected colonies were sent to BGI Genomics Co., Ltd. for sequencing; the sequencing primer was the above CG-catA-F/KP-kpdBD-R; a sequencing manner was throughput sequencing; an accurate strain was named as Escherichia coli EC005; the plasmid was named as pEC-1; and through expanding propagation, the plasmid was extracted to obtain a plasmid pEC-1.

(6) Genome DNA of Corynebacterium glutamicum ATCC13032 was taken as a template; the segment qsuB was amplified by using a primer qsuB-F/qsuB-R (as shown in SEQ ID NO. 50); and Corynebacterium glutamicum-derived qsuB with seamless cloning homologous arms in ligation with the plasmid was obtained.

Genome DNA of Corynebacterium glutamicum ATCC13032 was taken as a template; the segment aroB was amplified by using a primer aroB-F/aroB-R (as shown in SEQ ID NO. 51); and a Corynebacterium glutamicum-derived aroB segment with seamless cloning homologous arms in ligation with the qsuB and with RBS was obtained.

Genome DNA of Corynebacterium glutamicum ATCC13032 was taken as a template; the segment aroD was amplified by using a primer aroD-F/aroD-R (as shown in SEQ ID NO. 52); and a Corynebacterium glutamicum-derived aroD segment with seamless cloning homologous arms in ligation with the aroB and with RBS was obtained.

Genome DNA of Escherichia coli K-12 was taken as a template; the segment tkt was amplified by using a primer tkt-F/tkt-R (as shown in SEQ ID NO. 53); and a Corynebacterium glutamicum-derived tkt segment with seamless cloning homologous arms in ligation with the aroD and the plasmid and with RBS was obtained.

Primer sequences were as follows:

qsuB-F:
SEQ ID NO. 54
5′-CATATGCGTACATCCATTGCCACTG-3′;;
qsuB-R:
SEQ ID NO. 55
5′-CTAGTTTGGGATTCCCCGCTCG-3′;;
aroB-F:
SEQ ID NO. 56
5′-GGGGAATCCCAAACTAGAAGGAGGATATACATATGAGCGCAG
TGCAGATTTTCAAC-3′;;
aroB-R:
SEQ ID NO. 57
5′-CTTTTAGTGGCTGATTGCCTCATAAGC-3′;;
aroD-F:
SEQ ID NO. 58
5′-GCAATCAGCCACTAAAAGGAGGATATACATATGCCTGGAAAA
ATTCTC-3′;;
aroD-R:
SEQ ID NO. 59
5′-CTTTACGTGAGGACATATGTATATCCTCCTTCTACTTTTTGAGA
TTTGCCAGGATATCGACC-3′;;
tkt-F:
SEQ ID NO. 60
5′-GCAAATCTCAAAAAGTAGAAGGAGGATATACATATGTCCTCACG
TAAAGAGCTTG-3′;;
tkt-R:
SEQ ID NO. 61
5′-GGGGATCCTCTAGATTACAGCAGTTCTTTTGCTTTCGC-3′;.

A plasmid vector PXMJ19 was linearized with a primer PXM-1-1F/PXM-1-1R to obtain a vector PXM, wherein primer sequences were as follows:

PXM-1-1F:
SEQ ID NO. 62
5′-GTAATCTAGAGGATCCCCGGGTACC-3′;;
PXM-1-1R:
SEQ ID NO. 63
5′-CAATGGATGTACGCATATGTATATCCTCCTTGTCGACCTGCA
GGCATGC-3′;.

The gene amplification system and gene amplification procedures were the same as above; vectors qsuB, aroB, aroD, tkt and PXM were obtained; and 1.5 μl of qsuB, 1.5 μl of aroB, 1.5 μl of aroD, 1.5 μl of tkt and 4 μl of PXM of different sources and 10 μl of Gibson ligase were added into PCR tubes. Ligation and transformation manners were the same as above; several selected colonies were sent to BGI Genomics Co., Ltd. for sequencing; the sequencing primer was the above qsuB-F/tkt-R; a sequencing manner was throughput sequencing; an accurate strain was named as Escherichia coli EC006; the plasmid was named as pXM-1; and through expanding propagation, the plasmid was extracted to obtain a plasmid pXM-1.

The plasmids pEC-1 and pXM-1 were simultaneously electro-transformed into Corynebacterium glutamicum GM4 competent cells, and experimental steps were the same as above. The cells were uniformly mixed and coated onto an LBHIS solid medium plate containing 50 g/L of kanamycin and 5 g/L of chloromycetin; after the colonies grew, single colonies growing on the plate were selected to verify kana (primer kana-F/kana-R) and Cm (primer Cm-F/Cm-R) genes; and a strain of which the two genes were verified accurate was named as Corynebacterium glutamicum MA1.

Primer sequences were as follows:

kana-F:
SEQ ID NO. 64
5′-GTGAAACCAGTAACGTTATACGATGTCGC-3′;;
kana-R:
SEQ ID NO. 65
5′-CTCACTGCCCGCTTTCCAG-3′;;
Cm-F:
SEQ ID NO. 66
5′-CTTCACCGCCTGGCCCTG-3′;;
Cm-R:
SEQ ID NO. 67
5′-CGCCCGGAAGAGAGTCAATTCAG-3′;.

The Corynebacterium glutamicum MA1 was respectively subjected to shaking flask fermentation in a 50 mL system and fermentation tank feed verification in a 5 L system.

### \textcircled{1} Strain Activation

Single colonies of the Corynebacterium glutamicum MA1 were selected to be inoculated into an LBHIS fluid medium and cultured at 30° C. at 200 rpm for 12 h; and then the colonies were transferred into a fresh LBG fluid medium for secondary activation for 12 h, wherein the activated strain MA1 was used for growth test or fermentation.

### \textcircled{2} Conditions of Shaking Flask Fermentation

Conditions of a constant temperature shaker were 30° C. and 200 rpm. 50 mL fermentation liquor (a fermentation medium contained 50 μg/mL of kanamycin and 5 μg/mL of chloromycetin) was filled in a 250 mL baffled flask; initial OD₆₀₀ of inoculation was 0.5; three parallel experiments were conducted for each strain; an inducer IPTG (having a final concentration of 0.8 mM) was added within 3 h after fermentation; and changes in saccharide components and fermentation products were detected by an HPLC method.

### \textcircled{3} Fermentation Conditions of a 5 L Fermentation Tank

Conditions of the fermentation tank were 30° C. and 400 rpm. An initial pH value was 7.0. OD₆₀₀ of inoculation was 2.0 (an inoculation dose was about 5-10% of a fermentation volume). Aeration was conducted at 0.2 L/min. A filling volume of the fermentation tank was 50% of the total volume. An appropriate amount of a defoaming agent was added during initial fermentation. Whether the defoaming agent needed to be added was determined according to the fermentation conditions. The pH value was automatically regulated with 50% phosphoric acid and 25% ammonia water respectively during fermentation. An inducer IPTG (having a final concentration of 0.8 mM) was added within 12 h after fermentation; glucose (having a glucose concentration of 800 g/L) was automatically fed at a rate of 5 mL/h within 24 h after fermentation; and changes in saccharide components and fermentation products were detected by an HPLC method.

### Fermentation Product Detection:

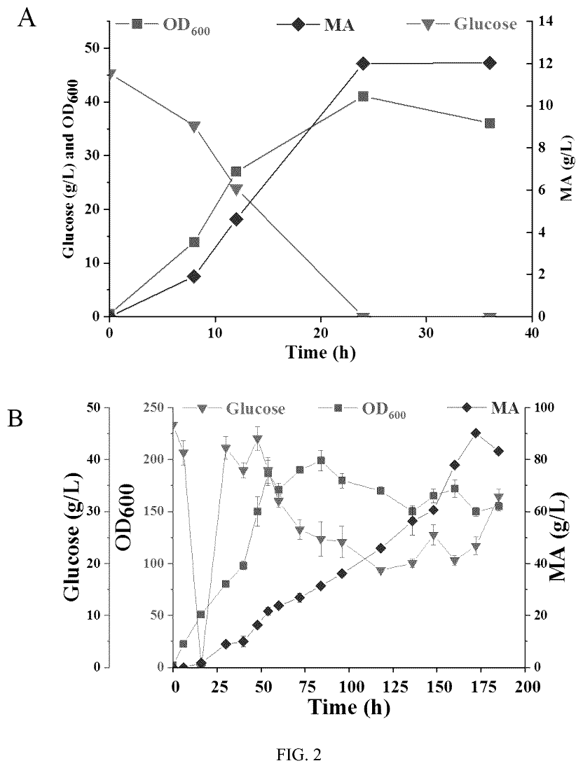

A liquid mass spectrometer was purchased from Agilent Technologies. The glucose was detected by a chromatographic column Aminex HPX-87H column; a differential detector UltiMate 3000 Variable Wavelength Detector was used; a mobile phase was 5 mM H₂SO₄; a column temperature was 65° C.; and a flow rate was 0.6 mL/min. The MA was detected by a chromatographic column Aminex HPX-87H column; a mobile phase was 5 mM H₂SO₄; an ultraviolet detector RefractoMax 520 was used; a wavelength of the ultraviolet light was 210 nm; a column temperature was 40° C.; and a flow rate was 0.5 mL/min. Data peak maps of different fermentation samples were subjected to content calculation according to a standard curve drawn from the standard sample, and results were shown as Table 1, Table 2 and FIG. 2.

TABLE 1
Shaking flask fermentation of a 50 mL system

| Time (h) | OD₆₀₀ | Glucose (g/L) | MA (g/L) |
|---|---|---|---|
| 0 | 0.50 | 45.30 | 0.00 |
| 8 | 13.90 | 35.61 | 1.91 |
| 12 | 27.00 | 23.90 | 4.63 |
| 24 | 41.05 | 0.00 | 11.99 |
| 36 | 36.05 | 0.00 | 12.04 |

TABLE 2
Fermentation tank feed experiment of a 5 L system

| Time (h) | OD₆₀₀ | Glucose (g/L) | MA (g/L) |
|---|---|---|---|
| 0 | 2.50 | 46.73 | 0.00 |
| 6 | 22.50 | 41.35 | 0.03 |
| 16 | 50.90 | 0.00 | 1.68 |
| 30 | 80.50 | 42.30 | 9.00 |
| 40 | 98.00 | 37.94 | 10.01 |
| 48 | 150.00 | 44.05 | 16.40 |
| 54 | 187.00 | 37.92 | 21.66 |
| 60 | 171.00 | 32.03 | 23.77 |
| 72 | 190.00 | 26.55 | 26.90 |
| 84 | 199.00 | 24.74 | 31.42 |
| 96 | 180.00 | 24.16 | 36.12 |
| 118 | 170.00 | 18.73 | 45.85 |
| 136 | 150.00 | 20.08 | 56.31 |
| 148 | 165.00 | 25.54 | 60.59 |
| 160 | 172.00 | 20.62 | 77.91 |
| 172 | 150.00 | 23.41 | 90.20 |
| 185 | 155.00 | 32.88 | 83.20 |

In the shaking flask fermentation of the 50 mL system, MA of 12.04 g/L was produced by the strain (FIG. 2A); and in the fermentation tank feed experiment of the 5 L system, MA of 90.2 g/L was produced by the strain (FIG. 2B).

### Embodiment 2

A construction method of a genetic engineering bacterium for de novo synthesis of cis,cis-muconic acid by taking glucose as a substrate includes the following steps:

Genome DNA of Escherichia coli EC869 was taken as a template; the segment kpdB was amplified by using a primer EC-kpdB-F/EC-kpdB-R (as shown in SEQ ID NO. 68); and an Escherichia coli EC869-derived kpdB segment with seamless cloning homologous arms in ligation with the plasmid was obtained.

Genome DNA of Escherichia coli EC869 was taken as a template; the segment kpdD was amplified by using a primer EC-kpdD-F/EC-kpdD-R (as shown in SEQ ID NO. 69); and a Corynebacterium glutamicum-derived kpdD segment with seamless cloning homologous arms in ligation with kpdB and the plasmid and with RBS was obtained.

Primer sequences were as follows:

EC-kpdB-F:
SEQ ID NO. 70
5′-CATATGAAACTGATCGTCGGGATGACAG-3′;;
EC-kpdB-R:
SEQ ID NO. 71
5′-CTTTTATTCATTCTCCTGAGAAAAATTCCGGGC-3′;;
EC-kpdD-F:
SEQ ID NO. 72
5′-TTCTCAGGAGAATGAATAAAAGGAGGATATACATATGATTT
GTCCACGTTGTGCCG-3′;;
EC-kpdD-R:
SEQ ID NO. 73
5′-GATTAGCGCTTACCTTCCGCCAG-3′;.
A plasmid vector pEC-1 was linearized with a
primer PEC-2-1F/PEC-2-1R to obtain a vector PECa,
wherein primer sequences were as follows:
PEC-2-IF:
SEQ ID NO. 74
5′-GGAAGGTAAGCGCTAATCTAGAGTCGACCTGCAGGCATG-3′;;
PEC-2-1R:
SEQ ID NO. 75
5′-CGACGATCAGTTTCATATGTATATCCTCCTTTTATTTTGCGCT
ACCCTGGTTTTTTTCC-3′;.

The gene amplification system and gene amplification procedures were the same as embodiment 1; vectors kpdB, kpdD and PECa were obtained; and 2 μl of kpdB, 2 μl of kpdD and 6 μl of PECa of different sources and 10 μl of Gibson ligase were added into PCR tubes. Ligation and transformation manners were the same as embodiment 1; several selected colonies were sent to BGI Genomics Co., Ltd. for sequencing; the sequencing primer was the above EC-kpdB-F/EC-kpdD-R; a sequencing manner was throughput sequencing; an accurate strain was named as Escherichia coli EC007; the plasmid was named as pEC-2; and through expanding propagation, the plasmid was extracted to obtain a plasmid pEC-2.

The plasmids pEC-2 and pXM-1 were simultaneously electro-transformed into Corynebacterium glutamicum GM4 competent cells, and experimental steps were the same as embodiment 1. Meanwhile, kana (primer kana-F/kana-R) and Cm (primer Cm-F/Cm-R) genes were verified; and a strain of which the two genes were verified accurate was named as Corynebacterium glutamicum MA2.

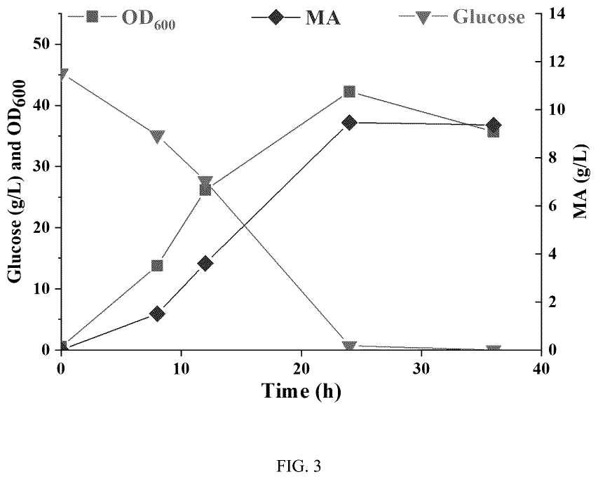

The Corynebacterium glutamicum MA2 was subjected to shaking flask fermentation verification in a 50 mL system, and a fermentation medium and fermentation operations were the same as embodiment 1. During shaking flask fermentation of the Corynebacterium glutamicum MA2 in the 50 mL system, MA of 9.4 g/L was produced by the strain (Table 3 and FIG. 3).

TABLE 3

| Time (h) | OD₆₀₀ | Glucose (g/L) | MA (g/L) |
|---|---|---|---|
| 0 | 0.50 | 45.30 | 0.00 |
| 8 | 13.80 | 35.10 | 1.51 |
| 12 | 26.15 | 27.65 | 3.60 |
| 24 | 42.25 | 0.70 | 9.47 |
| 36 | 35.70 | 0.00 | 9.37 |

### Embodiment 3

A construction method of a genetic engineering bacterium for de novo synthesis of cis,cis-muconic acid by taking glucose as a substrate includes the following steps:

Genome DNA of Pseudomonas putida KT2440 was taken as a template; the segment catA was amplified by using a primer PP-catA-F/PP-catA-R (as shown in SEQ ID NO. 76); and Pseudomonas putida KT2440-derived catA with seamless cloning homologous arms in ligation with the plasmid was obtained.

Primer sequences were as follows:

PP-catA-F:
SEQ ID NO. 77
5′-CATATGAGCAAGATTCTCACCACCGC-3′;;
PP-catA-R:
SEQ ID NO. 78
5′-CTTTTAGGCGAGATTGATGCCCAGG-3′;.

A plasmid vector pEC-1 was linearized with a primer PEC-3-1F/PEC-3-1R to obtain a vector PECb, wherein primer sequences were as follows:

PEC-3-1F:
SEQ ID NO. 79
5′-CATCAATCTCGCCTAAAAGGAGGATATACATATGACCGCAC
C-3′;;
PEC-3-1R:
SEQ ID NO. 80
5′-GGTGAGAATCTTGCTCATATGTATATCCTCCTTGAGCTCGA
ATTCTTCTGTTTCCTGTG-3′;.

The gene amplification system and gene amplification procedures were the same as embodiment 1.

The vectors catA and PECb were ligated in the Gibson ligation manner in embodiment 1. An accurate plasmid was obtained through the above same verification step; and the accurate plasmid was named as pEC-3.

The plasmids pEC-3 and pXM-1 were simultaneously electro-transformed into Corynebacterium glutamicum GM4 competent cells, and experimental steps were the same as embodiment 1. kana (primer kana-F/kana-R) and Cm (primer Cm-F/Cm-R) genes were continuously verified; and a strain of which the two genes were verified accurate was named as Corynebacterium glutamicum MA3.

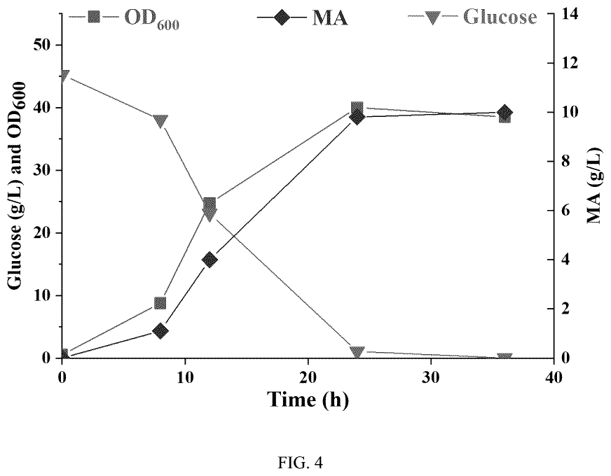

The Corynebacterium glutamicum MA3 was subjected to shaking flask fermentation verification in a 50 mL system, and a fermentation medium and fermentation operations were the same as embodiment 1. During shaking flask fermentation of the Corynebacterium glutamicum MA3 in the 50 mL system, MA of 10.0 g/L was produced by the strain (Table 4 and FIG. 4).

TABLE 4

| Time (h) | OD₆₀₀ | Glucose (g/L) | MA (g/L) |
|---|---|---|---|
| 0 | 0.50 | 45.25 | 0.00 |
| 8 | 8.77 | 38.07 | 1.10 |
| 12 | 24.71 | 23.09 | 4.00 |
| 24 | 40.00 | 1.07 | 9.80 |
| 36 | 38.50 | 0.00 | 9.99 |

### Embodiment 4

A construction method of a genetic engineering bacterium for de novo synthesis of cis,cis-muconic acid by taking glucose as a substrate includes the following steps:

Genome DNA of Escherichia coli K-12 was taken as a template; the segment aroG was amplified by using a primer aroG-F/aroG-R (as shown in SEQ ID NO. 81); and Corynebacterium glutamicum-derived aroG with seamless cloning homologous arms in ligation with the plasmid pXM-1 was obtained.

Primer sequences were as follows:

aroG-F:
SEQ ID NO. 82
5′-CATATGAATTATCAGAACGACGATTTACGCATCAAAG-3′;;
aroG-R:
SEQ ID NO. 83
5′-CTTTTACCCGCGACGCGCTTT-3′;.
A plasmid vector pXM-1 was linearized with a
primer PXM-2-1F/PXM-2-1R to obtain a vector
PXMa, wherein primer sequences were as follows:
PXM-2-1F:
SEQ ID NO. 84
5′-CGTCGCGGGTAAAAGGAGGATATACATATGTCCTCACGTAA
AG-3′;;
PXM-2-1R:
SEQ ID NO. 85
5′-GTCGTTCTGATAATTCATATGTATATCCTCCTTCTACTTTTTG
AGATTTGCCAGGATATCGACC-3′;.

The gene amplification system and gene amplification procedures were the same as embodiment 1.

The vectors aroG and PXMa were ligated in the Gibson ligation manner in embodiment 1. An accurate plasmid was obtained through the above same verification step; and the accurate plasmid was named as pXM-2.

The plasmids pEC-1 and pXM-2 were simultaneously electro-transformed into Corynebacterium glutamicum GM4 competent cells, and experimental steps were the same as embodiment 1. kana (primer kana-F/kana-R) and Cm (primer Cm-F/Cm-R) genes were continuously verified; and a strain of which the two genes were verified accurate was named as Corynebacterium glutamicum MA4.

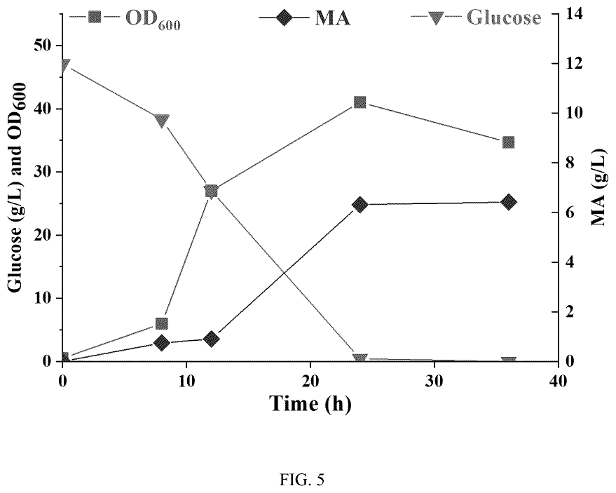

The Corynebacterium glutamicum MA4 was subjected to shaking flask fermentation verification in a 50 mL system, and a fermentation medium and fermentation operations were the same as embodiment 1. During shaking flask fermentation of the Corynebacterium glutamicum MA4 in the 50 mL system, MA of 6.4 g/L was produced by the strain (Table 5 and FIG. 5).

TABLE 5

| Time (h) | OD₆₀₀ | Glucose (g/L) | MA (g/L) |
|---|---|---|---|
| 0 | 0.50 | 47.06 | 0.00 |
| 8 | 6.00 | 38.29 | 0.74 |
| 12 | 27.00 | 27.01 | 0.90 |
| 24 | 41.00 | 0.44 | 6.32 |
| 36 | 34.67 | 0.00 | 6.42 |

Appearance time of an MA standard substance is 30.367 min; appearance time of a fermentation culture product of the genetic engineering strain prepared in embodiments 1-4 is 30.367 min and is consistent with the appearance time of an MA standard substance. Thus, it is proved that the fermentation product of the engineering strain is the target product, namely the MA.

The above description of the disclosed embodiments enables those skilled in the art to realize or use the present invention. Many modifications made to these embodiments will be apparent to those skilled in the art. General principles defined herein can be realized in other embodiments without departing from the spirit or scope of the present invention. Therefore, the present invention will not be limited to these embodiments shown herein, but will conform to the widest scope consistent with the principles and novel features disclosed herein.

What is claimed is:

1. A genetic engineering bacterium for de novo synthesis of cis,cis-muconic acid by taking glucose as a substrate, wherein the genetic engineering bacterium is a Corynebacterium glutamicum ATCC13032 serves as an original strain and modified with chassis microbes, and comprises recombinant Corynebacterium glutamicum for a cis,cis-muconic acid pathway construction module and an intermediate high-yield module;
modification of the chassis microbes is as follows: pyk, aroE, pcaG/H and catB genes are knocked out;
the cis,cis-muconic acid pathway construction module expresses protocatechuate decarboxylase genes, UbiX-like flavin prenyltransferase genes, 4-hydroxybenzoate decarboxylase, subunit D genes and catechol 1,2-dioxygenase genes;
the intermediate high-yield module expresses 3-dehydroquinate synthase genes, 3-dehydroquinate dehydratase genes, phosphate isomerase genes and transketolase genes.
the protocatechuate decarboxylase genes are Klebsiella pneumoniae-derived protocatechuate decarboxylase genes;
the UbiX-like flavin prenyltransferase genes comprise Escherichia coli-derived UbiX-like flavin prenyltransferase genes, and Klebsiella pneumoniae-derived UbiX-like flavin prenyltransferase genes;
the 4-hydroxybenzoate decarboxylasesubunit D genes comprise Escherichia coli-derived 4-hydroxybenzoate decarboxylasesubunit D genes, and Klebsiella pneumoniae-derived 4-hydroxybenzoate decarboxylasesubunit D genes;
the catechol 1,2-dioxygenase genes comprise Pseudomonas putida-derived catechol 1,2-dioxygenase genes and Corynebacterium glutamicum endogenous catechol 1,2-dioxygenase genes;
the 3-dehydroquinate synthase is a Corynebacterium glutamicum endogenous 3-dehydroquinate synthase gene;
the 3-dehydroquinate dehydratase genes are Corynebacterium glutamicum endogenous 3-dehydroquinate dehydratase genes;
the phosphate isomerase genes are Corynebacterium glutamicum endogenous phosphate isomerase genes;
a nucleotide sequence of the Klebsiella pneumoniae-derived protocatechuate decarboxylase genes is shown as SEQ ID NO. 37;
a nucleotide sequence of the Escherichia coli-derived UbiX-like flavin prenyltransferase genes is shown as SEQ ID NO. 68;
a nucleotide sequence of the Klebsiella pneumoniae-derived UbiX-like flavin prenyltransferase genes is shown as SEQ ID NO. 38;
a nucleotide sequence of the Escherichia coli-derived 4-hydroxybenzoate decarboxylase subunit D genes is shown as SEQ ID NO. 69;
a nucleotide sequence of the Klebsiella pneumoniae-derived 4-hydroxybenzoate decarboxylase, subunit D genes is shown as SEQ ID NO. 39;
a nucleotide sequence of the Pseudomonas putida-derived catechol 1,2-dioxygenase genes is shown as SEQ ID NO. 76;
a nucleotide sequence of the Corynebacterium glutamicum endogenous catechol 1,2-dioxygenase genes is shown as SEQ ID NO. 35;
a nucleotide sequence of the Corynebacterium glutamicum endogenous 3-dehydroquinate synthase gene is shown as SEQ ID NO. 51;
a nucleotide sequence of the Corynebacterium glutamicum endogenous 3-dehydroquinate dehydratase genes is shown as SEQ ID NO. 52;
a nucleotide sequence of the Corynebacterium glutamicum endogenous phosphate isomerase genes is shown as SEQ ID NO. 50;
a nucleotide sequence of the transketolase genes is shown as SEQ ID NO. 53.

2. The genetic engineering bacterium for de novo synthesis of cis,cis-muconic acid by taking glucose as the substrate according to claim 1, wherein
the protocatechuate decarboxylase genes are the Klebsiella pneumoniae-derived protocatechuate decarboxylase genes;
the UbiX-like flavin prenyltransferase genes are the Klebsiella pneumoniae-derived UbiX-like flavin prenyltransferase genes;
the 4-hydroxybenzoate decarboxylase subunit D genes are the Klebsiella pneumoniae-derived 4-hydroxybenzoate decarboxylase subunit D genes;
the catechol 1,2-dioxygenase genes are the Corynebacterium glutamicum endogenous catechol 1,2-dioxygenase genes;
the 3-dehydroquinate synthase Gene is the Corynebacterium glutamicum endogenous 3-dehydroquinate synthase gene;
the 3-dehydroquinate dehydratase genes are the Corynebacterium glutamicum endogenous 3-dehydroquinate dehydratase genes;
the phosphate isomerase genes are the Corynebacterium glutamicum endogenous phosphate isomerase genes

3. The genetic engineering bacterium for de novo synthesis of cis,cis-muconic acid by taking glucose as the substrate according to claim 2, wherein the intermediate high-yield module further expresses phospho-2-dehydro-3-deoxyheptonate aldolase genesa nucleotide sequence of the phospho-2-dehydro-3-deoxyheptonate aldolase genes is shown as SEQ ID NO. 81.

4. A construction method of a genetic engineering bacterium for de novo synthesis of cis,cis-muconic acid by taking glucose as a substrate, comprising the following steps:
(1) taking Corynebacterium glutamicum ATCC13032 as an original strain, and knocking out pyk, aroE, pcaG/H and catB genes to obtain modified Corynebacterium glutamicum;
(2) expressing the cis,cis-muconic acid pathway construction module and the intermediate high-yield module according to claim 1 in the modified Corynebacterium glutamicum.

5. An application of the genetic engineering bacterium according to claim 1 in production of cis,cis-muconic acid.

6. An application of the genetic engineering bacterium according to claim 1 in yield increase of the cis,cis-muconic acid.

7. A method for producing cis,cis-muconic acid, wherein fermentation is performed by utilizing the genetic engineering bacterium according to claim 1.


SEQUENCE LISTING (85 sequences)

SEQ ID NO: 1 - DNA, 1032 bp, Corynebacterium glutamicum
  source 1..1032: mol_type = genomic DNA
ctttttccagagctttttggaacagcttgccggtgacaccgtcaatatttccactgatgtacgtacgcccgtccacgtggcggaaagtcaaggtgccatcactcccctttggcccacgcttctttcctgtgaaggttggatccagttccttgatccagcgagtaatcagccctgcaagtgcggaggcttccatcagtgcctggccagcaatgcgcggtgtcagcttttcggcaatgcgatggtccaactcatcccacaactccggtttagcctgcttgactgcctgcataatgcgggccaggtacggaatccgaacgtggaagtgagtttctaccaccactcttaaaatgggcagcttcattaaagcccacatggcgttaatagcctggttgataaaatgcttggattcccccgtgacggttttcaactccatgacaagagtgctagcgtccgcctctgggtttgagatcagcaaagtccagcgctggatctccatttgcgccatggtgaagttgatgtgactctctggattatttgggtcctgatggctgaagtatgacatttccatgccccctaatggtttaaatttcccctttttaactcattagcacccaccctaaccacacaaatcaaaacaaacaagtgttctaacgaatatttgttctaattcttgaaaataaagataccgctctaccgaaactgcaggtagagcggttagaacctcaactatcccttgaggtaatactccagagcattggggtgttttgccagtggagtgatcttgacattcatgtatttgaacatcggaaagccactaagaattgcatgcagctcgtcgtggtcatcgacatcgaaaatggagtagtttgcatactcgccgacgactcgccagattgctttcatgattccacgggattgcaggtctccggagtaggccttttccttagcctggaaatctgccatcacatcggcgtccatggaatcagggaagacgacgtccatgcgtgctagaaacagcatcgataattctcc

SEQ ID NO: 2 - DNA, 1033 bp, Corynebacterium glutamicum
  source 1..1033: mol_type = genomic DNA
tttttctatggctttctggttaagtgggaaaagtcccgccacaagatgtggtcgggacttcaatgctagctaggtagaaaaactagtcttccttatccaggacgaatgggtagtgaacgtggttaccatcctcgccagtctcagggtgcaggaccagttctggcttcacagcggttgcaacgtcgttttcgacccactcgccaccctcgaagtaaagctgggtggtgatggtgcggtagcccgggtgggaaacgcgcaagtggaggtgggctgggcgccatgggtgcccaccgtaagactcaatgaaccaaccggttgggccatcatgagggatctggtaaggcgcaggctgcagggtcttgatcttgtagcggccttcctcatcggtaacgatggtgccacgcaggttccactctgggattccaggcgcgaactgggagtagtatccgtcctcatctgcgtgccagagctcaacttctgctccatcaagaccgttgccgtcgaggtcagtaacctgcccctcgaagatcagtggggtgcatgcgcggtccttgtcacgcattggcatttcagcatcccaagggagcttcggagagttctctacgtaataagggccttcgatggaacccttggttccggtgtagtcgtggcggttgtagttgatctcttcgatctcatgctcaacgaaaacgtccaaccacagtggccactcgccgtattctccaacgtcgatcatccactgcttgagcactgcgtactcttcgtaggtgacttcgtgcttgtgagcaacctgggcgatcgccgcgagcagatctacgtagatcgcgtttgcgcgttccttggaggtatcggaggaaacgcggtttgccttgaacttgtcagttgccttgttgcccgaatcgtgggctgttggatcaacgatctgttcagctgaagtcatgtcatactcctagagactttgggggtgaagtagggggtgcgagtcggatcactgcctagcggaaaaatctttgtgatcggctgaactcgataa

SEQ ID NO: 3 - DNA, 34 bp, synthetic construct
  source 1..34: mol_type = other DNA
ctagactttttccagagctttttggaacagcttg

SEQ ID NO: 4 - DNA, 48 bp, synthetic construct
  source 1..48: mol_type = other DNA
ccagaaagccatagaaaaaggagaattatcgatgctgtttctagcacg

SEQ ID NO: 5 - DNA, 31 bp, synthetic construct
  source 1..31: mol_type = other DNA
tttttctatggctttctggttaagtgggaaa

SEQ ID NO: 6 - DNA, 32 bp, synthetic construct
  source 1..32: mol_type = other DNA
ttatcgagttcagccgatcacaaagatttttc

SEQ ID NO: 7 - DNA, 35 bp, synthetic construct
  source 1..35: mol_type = other DNA
atcggctgaactcgataaaagcttggcactggccg

SEQ ID NO: 8 - DNA, 41 bp, synthetic construct
  source 1..41: mol_type = other DNA
caaaaagctctggaaaaagtctagaggatccccgggtaccg

SEQ ID NO: 9 - DNA, 23 bp, synthetic construct
  source 1..23: mol_type = other DNA
ctcaagcgtttgcgaaagaaacg

SEQ ID NO: 10 - DNA, 26 bp, synthetic construct
  source 1..26: mol_type = other DNA
gagtcagtgaacaggtaccatttgcc

SEQ ID NO: 11 - DNA, 1003 bp, Corynebacterium glutamicum
  source 1..1003: mol_type = genomic DNA
gctcaccgcgattttagagtggcgaaactctcattctttaccgttagcaaccctatggggtctcatcagctgcgccttaccttttgcggggccacttttctggattgttataagctactttaagaatagaaaaaaataaaagaatcaaagcgaagtgctatttacttgattcacccgttgataaatatttattcatatattgaagttctcggcatacattccccataaagctcataaactctgtgtcgtgacctctcagattattatgatgagttttcccttttccacgtaattgaattagcacgaagggcgattcccaaatgaccaatgggtcggatcgcctttttgctcatgctcagctcactggcgtacagcgcacagttacatttcgaccagatagcgcactggatgcactgtcgtgattacttacttctcagctatagacatactttcgctacttattctcagcgtcattatgctcatctgcagtgcgccagtcttattagtcatctttcgcagcaacagtcctgacgacctcatcgcagcactcatcctgacagcgttgttgggttttctcgctgtcaattttgtactccccacactcgcagacatcggctgcacgctgtgcgagctttaaaccagtaccccaaccagaaagtgactacgacatgagcgcaaaatcccctaccccacagcctccgtacgaaataccatcattccggaaaagacgcctaatcactaccggattattaacaatcacaacattgtggttcatctacttttatgtggatctagtaagcgccactacgctacttacgctgccggtgattatcgctgcaccattgatcatggtctacatctggtaccacgaattcacatactatgctcgtgtccgcaaaaacaataagctttacaaggctctctatgggcacctccaaaaccatcaacatcctgaaaatacggatactccacccatgtaaatagtcactaagatttagtcaca

SEQ ID NO: 12 - DNA, 1024 bp, Corynebacterium glutamicum
  source 1..1024: mol_type = genomic DNA
tccgagagccgagtaccgcttaagtctcaggcataaatcaagaactcagagacgatccggcaggacgaatgacatccacaggttcaggctgatccgtcacctcgatcacatattgaacctctgagtccgaaccatccgggaaagtcacttctgtacgaaaccacagttgaccggttcgatcatcaggcgccgtgaaggtgagatcgccagtcttctcatctatgagaatgattgcacccatcaactccgcattctcaattgagccgtaacgatctcggaccaccttcgatccctgtgggaccccaccaaccgctcctgtaccaccatccgtcaccctcaatgggatcttgacagtctgaccattgcggactttaggataacccgtgctttcaatgccgtagtaaatactgtcagccacataaaccactgaatggggtgtcactctatcaaatgacccatcagggtagaccaccttgacagtcactcgatctgtataagatcgtttcatttcaccaggggtgtagctgatccccgaagccccaagagtatgtcgaatctcccctgtatttgggtccaatcgagaagcgagcagaccatcatcaataataattgaaaacgaatcaccctcaagtaaaaatcgagtctcctctggtaattcagcaaggttttcaacagactgttcaaccgacttgggaaagactccactagaaccgagacgcgtttctaaggattcataccgtggttcatactgagcagccatagacacattttcatatgcaatcgcaacacccgtgccatttaccgttgctccgaacaacagtgaacatgacagtgcagcggtaaccaacgtgcgtgaacgtaccaacatacttctacaccactttctaaattagagatactctatgccacctcacagtataggtacaggatgtgtggcgttccctgccctcatggacggggttgctgcgtaccaactgccctacacattgcgagagccggataacgcttaagtctcaagcagaga

SEQ ID NO: 13 - DNA, 26 bp, synthetic construct
  source 1..26: mol_type = other DNA
gtcgctcaccgcgattttagagtggc

SEQ ID NO: 14 - DNA, 37 bp, synthetic construct
  source 1..37: mol_type = other DNA
gatgtgactaaatcttagtgactatttacatgggtgg

SEQ ID NO: 15 - DNA, 38 bp, synthetic construct
  source 1..38: mol_type = other DNA
gtcactaagatttagtcacatccgagagccgagtaccg

SEQ ID NO: 16 - DNA, 28 bp, synthetic construct
  source 1..28: mol_type = other DNA
tctctgcttgagacttaagcgttatccg

SEQ ID NO: 17 - DNA, 38 bp, synthetic construct
  source 1..38: mol_type = other DNA
cttaagtctcaagcagagagacctgcaggcatgcaagc

SEQ ID NO: 18 - DNA, 39 bp, synthetic construct
  source 1..39: mol_type = other DNA
taaaatcgcggtgagcgactctagaggatccccgggtac

SEQ ID NO: 19 - DNA, 1000 bp, Corynebacterium glutamicum
  source 1..1000: mol_type = genomic DNA
aacgttgacggtgatgccatcgatgctggttgcagccctgctcactgctgagtgagtgacagcagccaactcgcgcagcgttgcccactccgcgtgccagctgccggtgccgcgctgcaaacggcagtcgagggcgtcgaaaagcgttgcaagaaggccaggtgcccggcgcgcgtaaccgtcgcacgcaatcgcagcggccggattggctttgtggggcatcgcggagctgccgccgggggatttctcccgcaactcgccgacctcggtttgtgagtaaaacaccacgtcaccagcaattttgcgtaccacaccagcggccgttgccagcgccgacgcgatcgcagtgatcggcgtgcgatcggaatgccacacccactgcggatcaaaaaggcccaactcctcggccagcttcgcctgaatctcgaagccacgcgggtgcaccgccgtcatatttccgctggcaccgccatacgacaccggaaactccagcgcctccagggcgcgtgccgcattgtccaccgcaaccagccagccgccggtcagcgcgccgaacgtcgtcggcgtcgcgatctgccccaacgtgcgccccatgatcggggttgctttatgctccgcggtgagctcggccaaatctcgcgcaagctttttaagcttgtcgacgacctcccccaccccttccttcatgcacagcattaacgcagaatcaatgatgtcctggctcgttgcgccaatgtggatgcctgccggattgatggccttgaggtcagtgaccagcggaatgagcggattaccgccctcggctgcgcgacgggaaagctcctccacatccaactgataagaatcaatggtggccttcgccattgctgcgtgctcgggggcagctgcaaccgccaaagcggcctccacgacaagaagattctttaggaaagtctcgtcggaaaggctgctgaggtgttctccactaccagcaaggtcggagtataaagatcgggtcaat

SEQ ID NO: 20 - DNA, 1000 bp, Corynebacterium glutamicum
  source 1..1000: mol_type = genomic DNA
gcgagacctttctgcgtctagtgatatagatctcaaagtagggatgattgaggccgttgacaagtagttcgcggggcgaacatgtcagatcgcgcgcgaacattcgccaccagcgcaaaccccgaaggtcaggggttaactaggggtgtgcataagggtttagtcatccaatacctcgagtgaactgcggagcttttcaaaagtcttctccatccgtgcttccagagtctgcccccactgccccgtgggcatctcttgcgccaacggtccgacaggttcggttaccactgtgcgattgcacggcccaatccaggcgacccgacccatggaatcaatcgccgcctggatgcgcccatcagtcttcatattgtgatgatgcgcgcagaggcactgcaagttccaggccacagtcaacccaccctcaccgaacgggatcacgtgatcgatctggcagttgtcggcagacaccgtgcagcccggatgcctacaggtgccgtcccgccccatcacataaaggcgtagctcgggacttggggtgtagccattttccacacgagtggcgatcgtatccaggtcaatgaccttcgaggcagtcttacccagcttccgggactcctcttgactcaggaatcccacccccggaatccaggacacgccacccaccaggggcgtgtataccgcgctgacgattttcactttggtccgccccgccaggaaggtgaccagggcccgcgcgaggtcctcgcccttctgtttcactttttccagagctttttggaacagcttgccggtgacaccgtcaatatttccactgatgtacgtacgcccgtccacgtggcggaaagtcaaggtgccatcactcccctttggcccacgcttctttcctgtgaaggttggatccagttccttgatccagcgagtaatcagccctgcaagtgcggaggcttccatcagtgcctggccagcaatgcgcggtgtcagcttttcggcaatgcgatgg

SEQ ID NO: 21 - DNA, 21 bp, synthetic construct
  source 1..21: mol_type = other DNA
aacgttgacggtgatgccatc

SEQ ID NO: 22 - DNA, 29 bp, synthetic construct
  source 1..29: mol_type = other DNA
attgacccgatctttatactccgaccttg

SEQ ID NO: 23 - DNA, 44 bp, synthetic construct
  source 1..44: mol_type = other DNA
gagtataaagatcgggtcaatgcgagacctttctgcgtctagtg

SEQ ID NO: 24 - DNA, 22 bp, synthetic construct
  source 1..22: mol_type = other DNA
ccatcgcattgccgaaaagctg

SEQ ID NO: 25 - DNA, 35 bp, synthetic construct
  source 1..35: mol_type = other DNA
ttcggcaatgcgatgggacctgcaggcatgcaagc

SEQ ID NO: 26 - DNA, 29 bp, synthetic construct
  source 1..29: mol_type = other DNA
atcaccgtcaacgttgactctagaggatc

SEQ ID NO: 27 - DNA, 1000 bp, Corynebacterium glutamicum
  source 1..1000: mol_type = genomic DNA
caacagaggtgccgttgtcaaagaattcctccccctttaggcgtgagaccagcatgtccagatcctcacggactggaacgatcactgctacatcagcaagacccaaggttgctgcgcgtggacctgcacctgctgagttccaggtgttcatggccacatggtggtgatatccacctgcggaggcaaacagtgcattggggattgatgtggtggcatcaaaaccaatcttgtccacatagaattgacgggctcgctcgacgttgcctacctgaaggtgaacgtgtccgatgctcgcagggagaatagcggtgttgttcaaaacggtttcgctgagatgactctgaaggtactggtttggatccaagtagatggtgtccatgcggacatcgccgttgtctgcatacttccatgcacttcgagggcggtcaacatagagctcaatgccgtttccctcaggatcggtgaagtaaaaagcttcagagactaggtgatctgaagaacccacaaaacgtgagcgtgattcttgcgctgcgcggtaaacggtggcagccaagctctcttgggtatcgaagaggaaagcggtgtgataaaggccagcttgacgaggatctaccgcaggaagacctggggtagaaatcaggcgaagcagtggggtgccattgcgaccgagtacacggtgaacttcttttccgtgaactttttcttccattggttcaacgctaaggattgaagagtagtaactagtcataccctctaggtcgccgacgcgaaggctaactgcatccatgaaggtgtcgctattgatgctctgctcaagaagtgccatggtgtttcatcctttaagtgttggtggctgttttgctgttgaacaccattgttcaccaatttactttccgtgtcaagtaaattcttaaatgaggcggatttcccctgccctagccacgaaaaatgcgctgacaccacgtacagtgtcaacgcattttttgaaagatttaagcga

SEQ ID NO: 28 - DNA, 1001 bp, Corynebacterium glutamicum
  source 1..1001: mol_type = genomic DNA
gcccataagcctagtacgtcattccccctcaagccacaggtactaccattaattctcgatgaatcttgcgacccagagtgtgaacagacaatgttctacgcctctgacgtgcttaaatcagaaaaaaccgggcacccacaaaacaattcgggcactcggtttttctaccaaaaacatctcagccaggtcaggcctatggttgaaacctatcgcgttgagggcgtttctccatcaatgccctcccctgctttcggcaagggttttccatccggcgaagctacagcatctgctgcaacagacgctgcgaaagtcggatctacctcttcgggagtttccctacccttcttcaacaggaagaacacgatgatcgcgccggcaaacactactgcggagacgatggtgttgattcggatgccgccaataagcgtggcttcatcaacgcgcatttgttcaatccagaaacggcccaaggtataaccagctacgtagagtgcaaatactcggccatgtcccagcttgaatcgcttgtcagcccacatcaacaaagcgaagatcagtaggttccacaacagttcatagaggaatgttggatgaacagtagccattacttcaccggtggatgttcctgtcaccggtgcgaattttccattttcatctacccgatagtagatttccaaagcccatggaacggtagtttctgcaccgtagagctcctggttaaaccagttgcccagacgaccaattccctgcgccaggataactgcaggtgccacggcatctgcgaaaggtgcaagaggaagctttttgtaccggaagaatacggccactgccaggccaccgaggatcactgcaccccagatgcccagaccaccgttggtgattttgaaggcgtcgacggggttacaggtatcgcagaagtacttttggttgtcggtaatgacgtgataaatgcgtccaccgatgattccggcaggaactgccacgatcgctgcatcaaggac

SEQ ID NO: 29 - DNA, 33 bp, synthetic construct
  source 1..33: mol_type = other DNA
ctctagagtccaacagaggtgccgttgtcaaag

SEQ ID NO: 30 - DNA, 35 bp, synthetic construct
  source 1..35: mol_type = other DNA
ggctcgcttaaatctttcaaaaaatgcgttgacac

SEQ ID NO: 31 - DNA, 40 bp, synthetic construct
  source 1..40: mol_type = other DNA
gaaagatttaagcgagcccataagcctagtacgtcattcc

SEQ ID NO: 32 - DNA, 27 bp, synthetic construct
  source 1..27: mol_type = other DNA
caggtcgtccttgatgcagcgatcgtg

SEQ ID NO: 33 - DNA, 29 bp, synthetic construct
  source 1..29: mol_type = other DNA
catcaaggacgacctgcaggcatgcaagc

SEQ ID NO: 34 - DNA, 31 bp, synthetic construct
  source 1..31: mol_type = other DNA
ctctgttggactctagaggatccccgggtac

SEQ ID NO: 35 - DNA, 858 bp, Corynebacterium glutamicum
  source 1..858: mol_type = genomic DNA
atgacttcagctgaacagatcgttgatccaacagcccacgattcgggcaacaaggcaactgacaagttcaaggcaaaccgcgtttcctccgatacctccaaggaacgcgcaaacgcgatctacgtagatctgctcgcggcgatcgcccaggttgctcacaagcacgaagtcacctacgaagagtacgcagtgctcaagcagtggatgatcgacgttggagaatacggcgagtggccactgtggttggacgttttcgttgagcatgagatcgaagagatcaactacaaccgccacgactacaccggaaccaagggttccatcgaaggcccttattacgtagagaactctccgaagctcccttgggatgctgaaatgccaatgcgtgacaaggaccgcgcatgcaccccactgatcttcgaggggcaggttactgacctcgacggcaacggtcttgatggagcagaagttgagctctggcacgcagatgaggacggatactactcccagttcgcgcctggaatcccagagtggaacctgcgtggcaccatcgttaccgatgaggaaggccgctacaagatcaagaccctgcagcctgcgccttaccagatccctcatgatggcccaaccggttggttcattgagtcttacggtgggcacccatggcgcccagcccacctccacttgcgcgtttcccacccgggctaccgcaccatcaccacccagctttacttcgagggtggcgagtgggtcgaaaacgacgttgcaaccgctgtgaagccagaactggtcctgcaccctgagactggcgaggatggtaaccacgttcactacccattcgtcctggataaggaagactag

SEQ ID NO: 36 - DNA, 15 bp, synthetic construct
  source 1..15: mol_type = other DNA
aaggaggatatacat

SEQ ID NO: 37 - DNA, 1509 bp, Klebsiella pneumoniae
  source 1..1509: mol_type = genomic DNA
atgaccgcaccgattcaggatctgcgcgacgccatcgcgctgctgcaacagcatgacaatcagtatctcgaaaccgatcatccggttgaccctaacgccgagctggccggtgtttatcgccatatcggcgcgggcggcaccgtgaagcgccccacccgcatcgggccggcgatgatgtttaacaatattaagggttatccacactcgcgcattctggtgggtatgcacgccagccgccagcgggccgcgctgctgctgggctgcgaagcctcgcagctggcccttgaagtgggtaaggcggtgaaaaaaccggtcgcgccggtggtcgtcccggccagcagcgccccctgccaggaacagatctttctggccgacgatccggattttgatttgcgcaccctgcttccggcgcccaccaacacccctatcgacgccggccccttcttctgcctgggcctggcgctggccagcgatcccgtcgacgcctcgctgaccgacgtcaccatccaccgcttgtgcgtccagggccgggatgagctgtcgatgtttcttgccgccggccgccatatcgaagtgtttcgccaaaaggccgaggccgccggcaaaccgctgccgataaccatcaatatgggtctcgatccggccatctatattggcgcctgcttcgaagcccctaccacgccgttcggctataatgagctgggcgtcgccggcgcgctgcgtcaacgtccggtggagctggttcagggcgtcagcgtcccggagaaagccatcgcccgcgccgagatcgttatcgaaggtgagctgttgcctggcgtgcgcgtcagagaggatcagcacaccaatagcggccacgcgatgccggaatttcctggctactgcggcggcgctaatccgtcgctgccggtaatcaaagtcaaagcagtgaccatgcgaaacaatgcgattctgcagaccctggtgggaccgggggaagagcataccaccctcgccggcctgccaacggaagccagtatctggaatgccgtcgaggccgccattccgggctttttacaaaatgtctacgcccacaccgcgggtggcggtaagttcctcgggatcctgcaggtgaaaaaacgtcaacccgccgatgaaggccggcaggggcaggccgcgctgctggcgctggcgacctattccgagctaaaaaatattattctggttgatgaagatgtcgacatctttgacagcgacgatatcctgtgggcgatgaccacccgcatgcagggggacgtcagcattacgacaatccccggcattcgcggtcaccagctggatccgtcccagacgccggaatacagcccgtcgatccgtggaaatggcatcagctgcaagaccatttttgactgcacggtcccctgggcgctgaaatcgcactttgagcgcgcgccgtttgccgacgtcgatccgcgtccgtttgcaccggagtatttcgcccggctggaaaaaaaccagggtagcgcaaaataa

SEQ ID NO: 38 - DNA, 594 bp, Klebsiella pneumoniae
  source 1..594: mol_type = genomic DNA
atgaaactgattattgggatgacgggggccaccggggcaccgcttggggtggcattgctgcaggcgctgcgcgatatgccggaggtggaaacccatctggtgatgtcgaaatgggccaaaaccaccatcgagctggaaacgccctggacggcgcgcgaagtggccgcgctggcggacttttcccacagcccggcagaccaggccgccaccatctcatccggttcatttcgtaccgacggcatgatcgttattccctgcagtatgaaaacgcttgcaggcattcgcgcgggttatgccgaagggctggtgggccgcgcggcggacgtggtgctcaaagaggggcgcaagctggtgttggtcccgcgggaaatgccgctcagcacgatccatctggagaacatgctggcgctgtcccgcatgggcgtggcgatggtcccgccgatgccagcttactacaaccacccggagacggttgacgatatcaccaatcatatcgtcacccgggtgctggatcagtttggcctcgactatcacaaagcgcgccgctggaacggcttgcgcacggcagaacaatttgcacaggagatcgaataa

SEQ ID NO: 39 - DNA, 237 bp, Klebsiella pneumoniae
  source 1..237: mol_type = genomic DNA
atgatttgtccacgttgcgccgatgaaaagattgaagtcatggcgacctcgccggtgaaaggggtctggaccgtgtatcagtgccagcactgtctttacacctggcgagataccgagccgctgcgccgcaccagccgcgaacactatccggaagcgttccgcatgacgcagaaagatatcgatgaggcgccgcaggtgccgcacgtaccgccgctattgccggaagataagcgttaa

SEQ ID NO: 40 - DNA, 31 bp, synthetic construct
  source 1..31: mol_type = other DNA
catatgacttcagctgaacagatcgttgatc

SEQ ID NO: 41 - DNA, 28 bp, synthetic construct
  source 1..28: mol_type = other DNA
ctagtcttccttatccaggacgaatggg

SEQ ID NO: 42 - DNA, 56 bp, synthetic construct
  source 1..56: mol_type = other DNA
cctggataaggaagactagaaggaggatatacatatgaccgcaccgattcaggatc

SEQ ID NO: 43 - DNA, 63 bp, synthetic construct
  source 1..63: mol_type = other DNA
atcccaataatcagtttcatatgtatatcctccttttattttgcgctaccctggtttttttcc

SEQ ID NO: 44 - DNA, 30 bp, synthetic construct
  source 1..30: mol_type = other DNA
catatgaaactgattattgggatgacgggg

SEQ ID NO: 45 - DNA, 28 bp, synthetic construct
  source 1..28: mol_type = other DNA
ttattcgatctcctgtgcaaattgttct

SEQ ID NO: 46 - DNA, 53 bp, synthetic construct
  source 1..53: mol_type = other DNA
gcacaggagatcgaataaaaggaggatatacatatgatttgtccacgttgcgc

SEQ ID NO: 47 - DNA, 28 bp, synthetic construct
  source 1..28: mol_type = other DNA
gattaacgcttatcttccggcaatagcg

SEQ ID NO: 48 - DNA, 40 bp, synthetic construct
  source 1..40: mol_type = other DNA
cggaagataagcgttaatctagagtcgacctgcaggcatg

SEQ ID NO: 49 - DNA, 57 bp, synthetic construct
  source 1..57: mol_type = other DNA
gttcagctgaagtcatatgtatatcctccttgagctcgaattcttctgtttcctgtg

SEQ ID NO: 50 - DNA, 1857 bp, Corynebacterium glutamicum
  source 1..1857: mol_type = genomic DNA
atgcgtacatccattgccactgtttgtttgtccggaactcttgctgaaaagctgcgcgcagctgcagatgctggatttgatggtgtggaaatcttcgagcaggacttggtggtttccccgcattcggcagagcagattcgtcagcgggctcaggatttgggattaaccctggatctgttccagccgtttcgagatttcgaaggtgtggaagaagagcagtttctgaagaatctgcaccgcttggaagagaagttcaagctgatgaacaggcttggcattgagatgatcttgttgtgttccaatgtgggcaccgcgaccatcaatgatgatgaccttttcgtggagcagttgcatcgtgcagcagatttggctgagaagtacaacgtcaagattgcttatgaagcgttggcgtggggcaagtttgtcaatgattttgagcatgcgcatgcacttgtggagaaggtgaatcacaaggcgctgggaacctgcttggatacgttccatattctttcccgtggttgggaaaccgacgaggtggagaacatccctgcggagaagatcttctttgttcagttagcggatgcgccgaagctgagcatggacattttgtcctggtcgcgtcaccaccgtgttttccctggtgaaggcgatttcgatctggtgaaattcatggttcatctggccaagacgggttatgatggcccgatttctttggagatcttcaacgattccttccgcaaggccgaggttggtcgcaccgcgattgatgggttgcgttctttgcgttggttggaagatcagacctggcatgcgctaaatgctgaggatcgtccaagcgctcttgaactgcgtgcacttcctgaggtcgcggaacctgagggtgttgatttcattgagatcgccactggacgtttgggtgagaccattcgggttcttcatcaattgggtttccgcttgggtggtcatcactgcagtaagcaggattaccaggtatggacccagggcgatgtgcgcattgtggtgtgtgatcgtggggtcaccggggctccaaccacgatctctgcgatgggctttgacacccccgatccagaagctgctcatgcccgtgcggaattgctgcgggctcagacaattgatcgtccccacatcgagggcgaagttgacctaaaaggtgtgtacgcaccggatggggtggagctgtttttcgcggggccgagccccgatggaatgcccgagtggctgccggaattcggcgtcgaaaagcaagaagctggtctcattgaagccatcgaccacgtcaatttcgcccagccgtggcaacattttgatgaggcagtgctgttttacaccgcgctgatggcgttggagactgtgcgtgaggatgagttcccgagcccaattggtttggtgcgcaatcaggtgatgcgttcgccgaatgatgcggtgcggttgctgctcagcgtggcgccggaggacggtgagcagggagatttcctcaacgcggcctacccggagcacattgcgttggccacggcggacatcgtggcggtggctgaacgtgcgcgcaaacgaggcctggatttcttgcccgtcccagagaattactacgacgatgtgcaggcgcgttttgatttgccgcaggaattcttggacacactcaaggaaaaccacctgctttacgaccgcgacgagaacggcgaattcctccacttttacacccgcacgttgggcacgctgttcttcgaagtggtggaacgccgcggcggttttgcaggttggggcgaaacaaacgctccggtgcggttggcggcgcagtatcgtgaggtgcgggacctcgagcggggaatcccaaactag

SEQ ID NO: 51 - DNA, 1098 bp, Corynebacterium glutamicum
  source 1..1098: mol_type = genomic DNA
atgagcgcagtgcagattttcaacaccgtccacgtcaatggatcttccccctatgatgtccacattggttccggcctcaacgagctcattgttcagcgcgcagcggaatcaggcgcggagcaggtagcgattttgcaccagcccagcatggatgacattgcatccgagttggatgcagcactagtcgctgctggtttgaaggtcctgcaccttaatgttcccgatgcggaaaacggcaagtccttggaagtagcggggcagtgctgggatgaattgggtggcgcagcattcggccgccgcgatatcgtcatcggacttggtggcggtgctgccacagatctcgcgggattcgtcgctgctgcatggatgcgtggcgtgcgcgtcattcaggttccaaccaccttgttggccatggtggacgctgcggtgggcggcaagactggcatcaataccgccgcaggcaagaaccttgtgggcgcgttccacgagcctgacgcagtattcattgacaccgatcgcctagccaccctgcctgacgcggaaatcatcgcgggatccgccgaaatcatcaaaactggtttcatcgccgacccagaaatcctgcgcctttacgaaactgatcccgcagcctgcctgaagaaagaagtcgaaggctcccacctacctgaactgatttggcgctccgtcaccgtcaagggctccgtggtcggccaagacctcaaagaatctagcctgcgcgaaatcctcaactacggacacacctttgcccacgccgtcgaactccgcgaaaacttccgctggcgccacggcaatgccgttgcagtgggcatgatgttcatcgccaacctctcccacaagctcgggcttatcgacgcgcccctcctcgagcgccaccgctcaatcctggcggccatcggtctgcccacttcctacgaaggcggagccttcgacgagctttacgacggtatgacccgcgacaagaaaaaccgcgacggcaacatccgcttcgtcgcactgaccgccgtgggcgaggttacccgcattgaggggccctcaaaacaagatttacagagtgcttatgaggcaatcagccactaa

SEQ ID NO: 52 - DNA, 438 bp, Corynebacterium glutamicum
  source 1..438: mol_type = genomic DNA
atgcctggaaaaattctcctcctcaacggcccaaacctgaacatgctgggcaaacgcgagcctgacatttacggacacgacaccttggaagacgtcgtcgcgctggcaaccgctgaggctgcgaagcacggccttgaggttgaggcgctgcagagcaatcacgaaggtgagctaatcgatgcgctgcacaacgctcgcggcacccacatcggttgcgtgattaaccccggcggcctgactcacacttcggtggcgcttttggatgctgtgaaggcgtctgagcttcctaccgttgaggtgcacatttccaatccgcatgcccgtgaagagttccgccaccattcttacatttccctcgccgcggtctccgttatcgctggcgctggcatccagggttaccgtttcgcggtcgatatcctggcaaatctcaaaaagtag

SEQ ID NO: 53 - DNA, 1995 bp, Corynebacterium glutamicum
  source 1..1995: mol_type = genomic DNA
atgtcctcacgtaaagagcttgccaatgctattcgtgcgctgagcatggacgcagtacagaaagccaaatccggtcacccgggggcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaaagtggggttacaccgctgggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacgggccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgtcgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaa

SEQ ID NO: 54 - DNA, 25 bp, synthetic construct
  source 1..25: mol_type = other DNA
catatgcgtacatccattgccactg

SEQ ID NO: 55 - DNA, 22 bp, synthetic construct
  source 1..22: mol_type = other DNA
ctagtttgggattccccgctcg

SEQ ID NO: 56 - DNA, 56 bp, synthetic construct
  source 1..56: mol_type = other DNA
ggggaatcccaaactagaaggaggatatacatatgagcgcagtgcagattttcaac

SEQ ID NO: 57 - DNA, 27 bp, synthetic construct
  source 1..27: mol_type = other DNA
cttttagtggctgattgcctcataagc

SEQ ID NO: 58 - DNA, 48 bp, synthetic construct
  source 1..48: mol_type = other DNA
gcaatcagccactaaaaggaggatatacatatgcctggaaaaattctc

SEQ ID NO: 59 - DNA, 62 bp, synthetic construct
  source 1..62: mol_type = other DNA
ctttacgtgaggacatatgtatatcctccttctactttttgagatttgccaggatatcgacc

SEQ ID NO: 60 - DNA, 55 bp, synthetic construct
  source 1..55: mol_type = other DNA
gcaaatctcaaaaagtagaaggaggatatacatatgtcctcacgtaaagagcttg

SEQ ID NO: 61 - DNA, 38 bp, synthetic construct
  source 1..38: mol_type = other DNA
ggggatcctctagattacagcagttcttttgctttcgc

SEQ ID NO: 62 - DNA, 25 bp, synthetic construct
  source 1..25: mol_type = other DNA
gtaatctagaggatccccgggtacc

SEQ ID NO: 63 - DNA, 49 bp, synthetic construct
  source 1..49: mol_type = other DNA
caatggatgtacgcatatgtatatcctccttgtcgacctgcaggcatgc

SEQ ID NO: 64 - DNA, 29 bp, synthetic construct
  source 1..29: mol_type = other DNA
gtgaaaccagtaacgttatacgatgtcgc

SEQ ID NO: 65 - DNA, 19 bp, synthetic construct
  source 1..19: mol_type = other DNA
ctcactgcccgctttccag

SEQ ID NO: 66 - DNA, 18 bp, synthetic construct
  source 1..18: mol_type = other DNA
cttcaccgcctggccctg

SEQ ID NO: 67 - DNA, 23 bp, synthetic construct
  source 1..23: mol_type = other DNA
cgcccggaagagagtcaattcag

SEQ ID NO: 68 - DNA, 594 bp, Escherichia coli
  source 1..594: mol_type = genomic DNA
atgaaactgatcgtcgggatgacaggggctaccggtgcgcctcttggtgtggcattactgcaagcgctgcgggagatgccgaatgtcgagactcatctggtgatgtcgaagtgggcgaaaaccaccattgaactggaaacgccttacagcgcccgcgatgttgctgccctcgctgacttcagccataacccggcggatcaggcggcgaccatctcctcaggttcctttcgtacagacggcatgatcgttattccgtgcagtatgaaaacgctcgccggtatccgcgctggttacgctgatggcctggtagggcgcgcggcggacgtcgtgcttaaagaaggccgcaaactggtgctggtgccgcgtgaaatgccgcttagcaccatccatctcgaaaatatgctcgcactttcacgcatgggcgtggcgatggtgccgccgatgcctgccttttataaccatcccgaaacggtagatgacattgtccaccacgtggtagcccgcgtgctggatcaatttggccttgaacatccctacgccaggcgctggcaaggattgccgcaggcccggaatttttctcaggagaatgaataa

SEQ ID NO: 69 - DNA, 237 bp, Escherichia coli
  source 1..237: mol_type = genomic DNA
atgatttgtccacgttgtgccgatgaacagattgaagtgatggcgaaatcgccggtgaaagatgtctggacggtatatcagtgccagcattgcctttatacctggcgcgataccgaaccgctgcgccgtaccagccgcgaacattatcccgaagcgttccgcatgacgcagaaagatattgatgacgcgccaatggtgccgagcatcccgccgctgctggcggaaggtaagcgctaa

SEQ ID NO: 70 - DNA, 28 bp, synthetic construct
  source 1..28: mol_type = other DNA
catatgaaactgatcgtcgggatgacag

SEQ ID NO: 71 - DNA, 33 bp, synthetic construct
  source 1..33: mol_type = other DNA
cttttattcattctcctgagaaaaattccgggc

SEQ ID NO: 72 - DNA, 56 bp, synthetic construct
  source 1..56: mol_type = other DNA
ttctcaggagaatgaataaaaggaggatatacatatgatttgtccacgttgtgccg

SEQ ID NO: 73 - DNA, 23 bp, synthetic construct
  source 1..23: mol_type = other DNA
gattagcgcttaccttccgccag

SEQ ID NO: 74 - DNA, 39 bp, synthetic construct
  source 1..39: mol_type = other DNA
ggaaggtaagcgctaatctagagtcgacctgcaggcatg

SEQ ID NO: 75 - DNA, 59 bp, synthetic construct
  source 1..59: mol_type = other DNA
cgacgatcagtttcatatgtatatcctccttttattttgcgctaccctggtttttttcc

SEQ ID NO: 76 - DNA, 1440 bp, Pseudomonas putida
  source 1..1440: mol_type = genomic DNA
atgagcaagattctcaccaccgccagcggtgcacctgtagccgacaaccagaactcccgctccgccggcccacgcggcccgctgctgctcgacgacttccacctgatcgagaagctcgcccatttcaaccgtgagaacatccccgagcgccgggtacacgcgaagggttcgggcgcctatggcacttttaccgtaacccgcgacatcaccggttacaccagcgccaaactgttcgagcaggttggcaagcagaccgagacatttctgcgcttctctaccgtgggtggcgaacgtggttctgcggacacagagcgagaccctcgcggatttgccgttaagttctacaccgaggaaggtaactgggacatcgtgggtaacaacacgccagtgttcttcattcgtgatccgctcaagttcccggactttatccacacccagaaacggcacccgcagtccaacctgaaaaacgcccaaatattttgggattcctggtcgcactcgcccgaggcgctgcaccaagtcaccatcctgttctctgatcgtggcattccggacggctaccgccacatgcacggctttggcagccacacctacagcctgatcaatgcccagggtgagcgcacttgggtcaaatggcacttcaagacccagcagggcatcaagaaccttgctccggcagatgccgcacgtttggcaggtactgacccggactacgcccagcgcgacctgttcgaagccattgagcgaggtgattacccacgctggactgtctgcatccaggtgatgagcgaagccgaagccgcgagccgcgacgagaacccgttcgacgtgaccaaaacctggtcacaaaaggactacccgttgatcgaagtgggcgtgcttgagcttaaccgtaacccgctcaattacttcgccgaagtcgagcaggctgccttcgggccaagcaacatggtgccgggggtcggcctgtcgccagaccgcatgctccagggtcgcgtattcgcctacgcggacgcacaccgctaccgtgtgggtaccaaccaccaacagttgccggtgaatgcgccacgctgcccggtcaacagctaccagcgcgacggctccatggccactggcagctatggcagtgcgccgaactacgaacccaacagctacgccgcggcaccgaagcagtcgccacgtcacgcagagcccgcattggccctgaatggttcggccgaccgttacgatcaccgcgaggacgctgactactacagccacgccggcgcgctgttccgcctgatgagtgatgagcagaaggccctgctgatcagcaacatcgctggaaccatggcgggtgtcagcgaaaacgtcatccaacggcagttgcagtacttcttcaaggccgacccagcctacggagaaggcatcgccaagggcctgggcatcaatctcgcctaa

SEQ ID NO: 77 - DNA, 26 bp, synthetic construct
  source 1..26: mol_type = other DNA
catatgagcaagattctcaccaccgc

SEQ ID NO: 78 - DNA, 25 bp, synthetic construct
  source 1..25: mol_type = other DNA
cttttaggcgagattgatgcccagg

SEQ ID NO: 79 - DNA, 42 bp, synthetic construct
  source 1..42: mol_type = other DNA
catcaatctcgcctaaaaggaggatatacatatgaccgcacc

SEQ ID NO: 80 - DNA, 59 bp, synthetic construct
  source 1..59: mol_type = other DNA
ggtgagaatcttgctcatatgtatatcctccttgagctcgaattcttctgtttcctgtg

SEQ ID NO: 81 - DNA, 1053 bp, Corynebacterium glutamicum
  source 1..1053: mol_type = genomic DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 82 - DNA, 37 bp, synthetic construct
  source 1..37: mol_type = other DNA
catatgaattatcagaacgacgatttacgcatcaaag

SEQ ID NO: 83 - DNA, 21 bp, synthetic construct
  source 1..21: mol_type = other DNA
cttttacccgcgacgcgcttt

SEQ ID NO: 84 - DNA, 43 bp, synthetic construct
  source 1..43: mol_type = other DNA
cgtcgcgggtaaaaggaggatatacatatgtcctcacgtaaag

SEQ ID NO: 85 - DNA, 64 bp, synthetic construct
  source 1..64: mol_type = other DNA
gtcgttctgataattcatatgtatatcctccttctactttttgagatttgccaggatatcgacc